SEQUENCE LISTING (16 sequences)

SEQ ID NO: 1 - DNA, 2922 bp, Mus musculus
tcaagttgccattaacttccgcggcggccgccagctcggcggcggggaggtggcagcagctgcagccgccgcccgggaagtccgccgcccggcgcgcccgcgcggggagccgacccggcctcggcccgcaccgctggcccgcgctgtcccggttccacgcctgattctggcaggtgatctgtaagacagtgactgagtatggatcatttgaacgaggcaactcaggggaaagaacattcagaaatgtctaacaatgtgagtgatccgaagggtccacccgccaagattgcccgcctggagcagaacgggagccctctaggaagaggaaggcttgggagcacaggtggaaagatgcagggagtgcctttaaaacactcgggccatctcatgaaaaccaaccttaggaaaggaaccatgttaccagttttctgcgtggtggaacattatgaaaacgccattgagtatgattgcaaggaggagcacgcggaatttgtattggtgagaaaggatatgcttttcaaccagctgatagagatggcgttgctgtctctaggctattcacacagctctgctgcccaagccaaagggctcatccaggttgggaagtggaatccagttccactgtcgtatgtgacagatgcccctgatgccacggtggcaaacatgcttcaagatgtgtatcatgtggtcaccctcaaaattcagttacacagttgccctaaactagaagacttgcctcctgaacaatggtcgcacaccacagtaaggaatgctctgaaggacttactgaaagatatgaaccagagttcgttggccaaggagtgccccctttcacagagcatgatctcctccattgtgaacagcacgtactatgcaaatgtctcagcagcaaaatgtcaagaatttggaaggtggtacaaacatttcaagaagacaaaggatatgatggttgagatggatagtctgtctgaactatcccagcaaggtgccaaccacgtcaattttggccagcagcctgtcccaggaaacacagctgagcagcctccatcccctgcgcagctctctcacggcagtcagccctctgtccggacccctcttccgaacctgcaccctgggcttgtgtcaacaccgatcagtcctcagctggtcaaccaacagctggtgatggctcagttgctgaaccagcagtatgcagtgaacagactcttagcccagcagtccttaaaccaacagtacttgaaccaccctccccctgtcagtaggtctatgaacaagcctttggagcagcaagtttccacaaacacggaggtctcttctgaaatctaccagtgggtgcgggatgaactgaaacgagccggaatctcacaggcagtatttgcacgcgtggcttttaaccgaactcagggattgctttctgaaatcctccgaaaggaagaggaccccaagactgcatcccagtctctgctggtaaaccttcgggctatgcagaatttcttacagttgccggaagccgaaagagaccggatataccaggatgagagggaaaggagcttgaacgcagcctcagccatgggtcctgccccgctgctgagcacaccacccagccgccctccccaggtgaaaacagctacccttgccactgagagaaatgggaagccagagaacaatactatgaacattaatgcctccatttatgatgagattcagcaggaaatgaagcgtgctaaagtgtcccaagcaccgtttgcaaaggttgccgccacaaaaagccagggatggctgtgtgagctgttgcgctggaaagaagatccttctccagaaaacaggaccctgtgggagaacctgtcgatgatccgaagattcctcagtctgcctcagcctgagcgcgatgccatctatgagcaggagagcaatgctgtgcatcaccatggcgacagacctccccacatcatccacgttccagcagaacagattcagcagcagcaacagcagcagcagcaacagcagcagcagcagcagccaccgccgccaccgccgcagccacagccacagccccaggcaggccccagactccccccacggcagcccaccgtggcctcctccgcggagtccgatgaggaaaaccggcagaagaccaggccacgaaccaaaatttccgtggaagccctggggatccttcagagtttcatccaagatgtgggcctgtacccagatgaagaggctatccagactctgtctgcacagctggacctcccgaagtacaccatcatcaagttctttcagaaccagcggtactaccttaagcaccatggcaagctgaaggacaactccggcttggaggtggatgtggccgagtacaaagatgaggagttgcttaaggatttggaagagagcgtccaggataaaaacgccaacacccttttctcagtgaaactagaggaagagctgtcggtggaagggagcacagacgttaatgccgacttgaaagactgagagaacagtattcttttcagccacaccaccggtatttcctaacaacatgagaagtccaccttgtgttcactcagacaaaccttcattgtttattgtttggccaatgaatcttcggaaacttgcacaaacaggaaggaagttggaaggacaggacagccagcactcaaggttttactgtgttttacaaaactgcttggcagccccgggtgaagcgtcaaggactgtttggtagaatttgtgttcaagggctgcacccaggtgttgtaccctgtcagcatgataccagaaattggttttccttttgattattattattctggagcctcaaataagcattataacttctgtggttatgattgccttttctttagtaattcttctgtaacccgccacactagtctttggaaactggccccttattttaaagag

SEQ ID NO: 2 - protein, 764 aa, Mus musculus
MDHLNEATQGKEHSEMSNNVSDPKGPPAKIARLEQNGSPLGRGRLGSTGGKMQGVPLKHSGHLMKTNLRKGTMLPVFCVVEHYENAIEYDCKEEHAEFVLVRKDMLFNQLIEMALLSLGYSHSSAAQAKGLIQVGKWNPVPLSYVTDAPDATVANMLQDVYHVVTLKIQLHSCPKLEDLPPEQWSHTTVRNALKDLLKDMNQSSLAKECPLSQSMISSIVNSTYYANVSAAKCQEFGRWYKHFKKTKDMMVEMDSLSELSQQGANHVNFGQQPVPGNTAEQPPSPAQLSHGSQPSVRTPLPNLHPGLVSTPISPQLVNQQLVMAQLLNQQYAVNRLLAQQSLNQQYLNHPPPVSRSMNKPLEQQVSTNTEVSSEIYQWVRDELKRAGISQAVFARVAFNRTQGLLSEILRKEEDPKTASQSLLVNLRAMQNFLQLPEAERDRIYQDERERSLNAASAMGPAPLLSTPPSRPPQVKTATLATERNGKPENNTMNINASIYDEIQQEMKRAKVSQAPFAKVAATKSQGWLCELLRWKEDPSPENRTLWENLSMIRRFLSLPQPERDAIYEQESNAVHHHGDRPPHIIHVPAEQIQQQQQQQQQQQQQQQPPPPPPQPQPQPQAGPRLPPRQPTVASSAESDEENRQKTRPRTKISVEALGILQSFIQDVGLYPDEEAIQTLSAQLDLPKYTIIKFFQNQRYYLKHHGKLKDNSGLEVDVAEYKDEELLKDLEESVQDKNANTLFSVKLEEELSVEGSTDVNADLKD

SEQ ID NO: 3 - DNA, 3782 bp, Homo sapiens
ttctgcccttcccccgcttaggggggcgggggtaggggaaaggaaaataatacaatttcaggggaagtcgccttcaggtctgctgcttttttattttttttttttaattaaaaaaaaaaaggacatagaaaacatcagtcttgaacttctcttcaagaacccgggctgcaaaggaaatctcctttgtttttgttatttatgtgctgtcaagttttgaagtggtgatctttagacagtgactgagtatggatcatttgaacgaggcaactcaggggaaagaacattcagaaatgtctaacaatgtgagtgatccgaagggtccaccagccaagattgcccgcctggagcagaacgggagcccgctaggaagaggaaggcttgggagtacaggtgcaaaaatgcagggagtgcctttaaaacactcgggccatctgatgaaaaccaaccttaggaaaggaaccatgctgccagttttctgtgtggtggaacattatgaaaacgccattgaatatgattgcaaggaggagcatgcagaatttgtgctggtgagaaaggatatgcttttcaaccagctgatcgaaatggcattgctgtctctaggttattcacatagctctgctgcccaggccaaagggctaatccaggttggaaagtggaatccagttccactgtcttacgtgacagatgcccctgatgctacagtagcagatatgcttcaagatgtgtatcatgtggtcacattgaaaattcagttacacagttgccccaaactagaagacttgcctcccgaacaatggtcgcacaccacagtgaggaatgctctgaaggacttactgaaagatatgaatcagagttcattggccaaggagtgccccctttcacagagtatgatttcttccattgtgaacagtacttactatgcaaatgtctcagcagcaaaatgtcaagaatttggaaggtggtacaaacatttcaagaagacaaaagatatgatggttgaaatggatagtctttctgagctatcccagcaaggcgccaatcatgtcaattttggccagcaaccagttccagggaacacagccgagcagcctccatcccctgcgcagctctcccatggcagccagccctctgtccggacacctcttccaaacctgcaccctgggctcgtatcaacacctatcagtcctcaattggtcaaccagcagctggtgatggctcagctgctgaaccagcagtatgcagtgaatagacttttagcccagcagtccttaaaccaacaatacttgaaccaccctccccctgtcagtagatctatgaataagcctttggagcaacaggtttcgaccaacacagaggtgtcttccgaaatctaccagtgggtacgcgatgaactgaaacgagcaggaatctcccaggcggtatttgcacgtgtggcttttaacagaactcagggcttgctttcagaaatcctccgaaaggaagaggaccccaagactgcatcccagtctttgctggtaaaccttcgggctatgcagaatttcttgcagttaccggaagctgaaagagaccgaatataccaggacgaaagggaaaggagcttgaatgctgcctcggccatgggtcctgcccccctcatcagcacaccacccagccgtcctccccaggtgaaaacagctactattgccactgaaaggaatgggaaaccagagaacaataccatgaacattaatgcttccatttatgatgagattcagcaggaaatgaagcgtgctaaagtgtctcaagcactgtttgcaaaggttgcagcaaccaaaagccagggatggttgtgcgagctgttacgctggaaagaagatccttctccagaaaacagaaccctgtgggagaacctctccatgatccgaaggttcctcagtcttcctcagccagaacgtgatgccatttatgaacaggagagcaacgcggtgcatcaccatggcgacaggccgccccacattatccatgttccagcagagcagattcagcaacagcagcagcaacagcaacagcagcagcagcagcagcaggcaccgccgcctccacagccacagcagcagccacagacaggccctcggctccccccacggcaacccacggtggcctctccagcagagtcagatgaggaaaaccgacagaagacccggccacgaacaaaaatttcagtggaagccttgggaatcctccagagtttcatacaagacgtgggcctgtaccctgacgaagaggccatccagactctgtctgcccagctcgaccttcccaagtacaccatcatcaagttctttcagaaccagcggtactatctcaagcaccacggcaaactgaaggacaattccggtttagaggtcgatgtggcagaatataaagaagaggagctgctgaaggatttggaagagagtgtccaagataaaaatactaacacccttttttcagtgaaactagaagaagagctgtcagtggaaggaaacacagacattaatactgatttgaaagactgagataaaagtatttgtttcgttcaacagtgccactggtatttactaacaaaatgaaaagtccaccttgtcttctctcagaaaacctttgttgttcattgtttggccaatgaatcttcaaaaacttgcacaaacagaaaagttggaaaaggataatacagactgcactaaatgttttcctctgttttacaaactgcttggcagccccaggtgaagcatcaaggattgtttggtattaaaatttgtgttcacgggatgcaccaaagtgtgtaccccgtaagcatgaaaccagtgttttttgttttttttttagttcttattccggagcctcaaacaagcattataccttctgtgattatgatttcctctcctataattatttctgtagcactccacactgatctttggaaacttgccccttatttaaaaaaaaaaaagaaaaaaaagagtttgttactctattgtatgttacaaaagaactatagactgtggaatgcagtttaaagatgacatatgccaacaaatgccttgtattatatggcactgccgtaattcaaatttgtttttattttggaaataaaagttcactgtacttttttttcattctcattgttacatgattttttaaaaaaaggaaaagaaaatgtgaaacacaatttagtcctcattatttatttgtagatcctgcagcatcatgttgtaattaattttttggaagtttccgttaaatgtaatattgcttctcttgttaccatactgattcttttctatttataaatgtattttgatgggcagtaaaacaaagtgtcttaaaagttttaaatagagaaaatgtgctttacacagttgcctataaaaagtgctctatgttatccaagcaattcatactataagcttcactcttattgttgtatgcaatttttactatcatgcaaataagcttaggtaaataaaactaatagatcaccttagaaaattatgcaattaatgtgaaaataattgatgtttgcaatgtgtcttcctttggtttacaatcaattttaaagctacatctgtataaaatttctgtataaaggtgtatttcttttttatgagtttatggctatgaaaacagctattttgttacagctggctgtttttataagtgtatcacaattttctttatgcagaaatgttctgactaggagtggttattgactgtaactacacaattaaaattgtttgtatcgtaaaaaaaaaaaaaaaaaa

SEQ ID NO: 4 - protein, 763 aa, Homo sapiens
MDHLNEATQGKEHSEMSNNVSDPKGPPAKIARLEQNGSPLGRGRLGSTGAKMQGVPLKHSGHLMKTNLRKGTMLPVFCVVEHYENAIEYDCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSSLAKECPLSQSMISSIVNSTYYANVSAAKCQEFGRWYKHFKKTKDMMVEMDSLSELSQQGANHVNFGQQPVPGNTAEQPPSPAQLSHGSQPSVRTPLPNLHPGLVSTPISPQLVNQQLVMAQLLNQQYAVNRLLAQQSLNQQYLNHPPPVSRSMNKPLEQQVSTNTEVSSEIYQWVRDELKRAGISQAVFARVAFNRTQGLLSEILRKEEDPKTASQSLLVNLRAMQNFLQLPEAERDRIYQDERERSLNAASAMGPAPLISTPPSRPPQVKTATIATERNGKPENNTMNINASIYDEIQQEMKRAKVSQALFAKVAATKSQGWLCELLRWKEDPSPENRTLWENLSMIRRFLSLPQPERDAIYEQESNAVHHHGDRPPHIIHVPAEQIQQQQQQQQQQQQQQQAPPPPQPQQQPQTGPRLPPRQPTVASPAESDEENRQKTRPRTKISVEALGILQSFIQDVGLYPDEEAIQTLSAQLDLPKYTIIKFFQNQRYYLKHHGKLKDNSGLEVDVAEYKEEELLKDLEESVQDKNTNTLFSVKLEEELSVEGNTDINTDLKD

SEQ ID NO: 5 - DNA, 5299 bp, Mus musculus
cgcccccatcatcataacaaccatctccgaaccagaagaagacaccctgacccaggaccttaaacgctaggaccggggaagaggaaaaggaaaggggaaggaggaaagagaaaggaagactaggacaacactacaattcaccaaaaactttccaccctggattctctacttttgctccatggacagagccccagccagccaagtttcagacagaccgtgagcagtccctgcgcgttttattgcgacctgccggtgggaactttgtctccaaatcggagcagcatggagcggcggagcgagagcccgtgtcttcgggacagccccgaccgaagaagcggcagccccgacgtcaaggggcctcccccggtgaaggtggcccggctggagcagaacggcagccccatgggagcccgcgggaggcccaacggcgccgtggccaaggccgtgggaggtttgatgattccagttttctgtgtggtggagcagttggatggctctcttgaatacgacaaccgagaagagcacgctgagttcgtcttggtgcggaaagatgtgctttttagccagctggtggagaccgcgctcctggccctggggtattcccacagctctgcagcgcaggcccaaggaataatcaagctagggaggtggaaccccctccccctcagttatgtgacagacgcccctgatgcgactgtggccgacatgctgcaagatgtctatcacgttgtgacgctgaagatccaattacaaagttgttcaaagttggaagacttgcctgcggagcaatggaaccacgccaccgtccgcaatgccttaaaggaactgctcaaagaaatgaaccagagcacattagccaaagaatgccctctctcccagagtatgatttcatccattgtaaatagcacatactatgccaatgtgtcagcaaccaagtgccaggagtttgggagatggtacaaaaagtataagaagataaaagtggaaagagtggagcgagagaacctttcagactattgtgttctgggccagcggccaatgcatttaccaaatatgaaccagctggcatccctgggcaaaaccaacgaacagtctcctcatagccaaatccaccacagtactccaatccgaaaccaagtgcccgcactccagcccatcatgagccctggtcttctctcaccgcagctcagtcctcagcttgtcaggcagcaaatagccatggcccatctgataaaccaacagatagccgttagccgactcctggctcaccagcatcctcaagccatcaaccagcagttcttgaaccacccacccattcccagagcagttaagccagagccaacaaactcctctgtggaagtctctcctgatatctaccagcaagttagagatgagttgaagagggctagtgtgtctcaagctgtctttgcaagagtggcattcaaccgcacacagggattattgtcagagatactgcgtaaggaagaagatcccaggactgcgtctcagtctcttctagtaaacctgagggccatgcagaacttcctcaacctgcctgaagtggagcgtgatcgcatttaccaggatgagcgagagaggagcatgaaccccaatgtgagcatggtctcctctgcctctagcagtcccagctcctcccgaaccccacaggccaaaacctcgacaccgacaacagacctccctattaaggtggacggcgccaacgtcaacatcacagctgccatttatgacgagatccaacaggagatgaaaagagccaaggtgtctcaagccctgtttgccaaagtggctgcaaacaaaagtcagggctggctttgcgaactgcttcgttggaaggagaaccccagcccagaaaaccgcaccctttgggagaatctctgcaccatccgccgtttcctgaatcttccccaacatgagcgggatgtgatctatgaggaagaatctcgacatcaccacagtgaacgcatgcagcatgtggtccagctcccacctgagcccgtgcaggtccttcatcgacagcagtcccagccaactaaggagagctcccctcccagagaagaagcacccccaccgcctcctccaacagaagacagctgtgccaaaaagcctcggtctcgcacaaagatctctttggaagccctgggcatccttcaaagcttcatccatgatgtaggcctctatcccgaccaggaagccatccacacactctccgcccagctggatctccccaaacacaccatcatcaagttcttccagaaccagaggtaccacgtgaagcaccatggcaagctgaaggagcacctgggctccgccgtggacgtggcagagtataaggacgaggagctgctgaccgagtccgaggagaatgacagtgaggaaggctcggaggagatgtacaaggtggaggccgaggaagagaatgccgacaagagcaaggctgcaccggccgaaaccgaccagagataatgtgaactccgcaggcagagcaatagatgggtccaaggactttctgttttagtttctttcaagttcctctcggctttgttttattttgttttggttttgtctgttttatttttcttacccttaatggcattcctctgttgcacaggatagcctatagactgagtaagctcagagtttctgaatcagacgtcaccttggcaaagacactaagtgttaaacttttacaattgactcgaccatagtaattctagtcacgggcgaccctgccgtcattcctgccaacttatggaagcgataccaggcaaattccaggatgggaagacccacgagataaatgaaggaagctgcaagtgtgtatatgtatatgtacatatctctatatttacatatatatattaaaattgcatgggacagagaactttacaatccgaaggactagactgtgaaatgagttcttaaagaaaagacttgtttatgtataaaaactaataccacttcacagtgagtcactttgactttgagaaactgcagcctgctctcagggcgggataactatttttgaattcctatttattttgtgtgtttgtccctgatttttttttttttaattctatggcttcctatctggcagcttttaatgggtaatttctgaggtatgtatttaacaaaataacactgccaaaaagatgagagacaggaggaaagaagagagtaaagtgggagaaaatcagggcatgtcaaaaaaaccaacataagaccaattcctccagaggacataatgcacaccttaagacaaggactcagcgccgtgaccctccacactccattcccgagcatgccagcactgtatcgagcggatgccttctgtggaatcccgagcccagttcagtgcagaggaaacttgacctttagcgcagtctcaatgagtgacgttttgacgttggacctatggaatccatgctatgagccacattttgttgtgaagtttatttactcgcttgtggcttcaaatcttaaaattattaagcctgctcatttaatattgttagggtgttaccctttccttcctttcctttctttctttctttctttctttctttctttctttctttctttctttctttctttctttttttttttctggatttcttttgttcttgttcttgttttggttttggtttggtttgttttaaatagaacatagtttaatgtaatgttaggttagagaaagaagtttctgcccggttagaggggctcccgctcagataactccaccctgccctctccccgagtgttcctactctgcttccgtttagacttcctcttcttcacaaatccccttcactttagccagacctctgcagaccctgagctcacgcgtctccacccttcccctgccacgacagccctggcaggaacctggtttggaatcatgcacctccaggcaataaagtaagtgaagcgatttcagcagagtcacctactcatcggaaaaggaaataattgtcccagtgccccacatagcgcttaccttacgtaaagcagaactaatgccaacgggctgttttgtggaacaagcgttaaagctgcaatctaccacagtttgccaaatctcctccagtttcctattagaaacagtagcattaccttccagttccagtgacagcgattgcaagaggagacctcccttccttcgggaccggcctccggaacccactccaggcttgagatggccactcagcagtcccgcacggttggattttattttcttttgttttgtttttgagttgtatcctcatgcaaccttgtcaaagacctcatgcaatatcactttgaaatttattttctgtttactacacaaacattgtaatataactgttaatactatttatatatttgaaaggtataagaggtaggagtttaaaaaaaaaacctctatgtgtagatattaactcagagcttacactatacagggagaagacacgttgcaatacaagctaattctagctgcttagtaacctctggagttttcaaagggacatttttcctgtactttttcaaataatgatgtttaaaacagttacccttgacatgagcgtcatgcagctttgcaaaaggatggctgtttgcaactgaggcctgggcccgcccgtcccctttctgtagatgctgcagctttaatccgagagtccacagttgctgcttcctgatctccgagttactctttccaaattgtcttctgacgctgttgctgaagtcactctgtacacatcctagacaccgatttttttgccaagctcttacaggtggttcatcgatcgatggcatccgcatttggtatcttttacacttcaaccaaaaatttattaggtatttttcaatgctaagtcttgccttttattttttaatttcactgccaagtttgcagtggttctaagtgaatctgtgggcattttagcctgtggtcttgccagatctttgcgaattacaatgcatatatgtctatttattcaatatctgtcatataatatctatttggaagaagaaactttctcttgtagtgcctcttgacaaagcacaatttcccgcctttttttttttttttgtgaaatgaagaaaaaaacaaattgtgttttattgcagtatcaataatgtgaataaggattaacatattgtaaatgttcctttttccatgtaaatcaactatctttgttatcactaagtgataatttttaacttatgtgcattgttaggctgttagaattttttggttgttaaataaaacgcattcaataaatctgaaaaaaaaaaaaaa

SEQ ID NO: 6 - protein, 733 aa, Mus musculus
MERRSESPCLRDSPDRRSGSPDVKGPPPVKVARLEQNGSPMGARGRPNGAVAKAVGGLMIPVFCVVEQLDGSLEYDNREEHAEFVLVRKDVLFSQLVETALLALGYSHSSAAQAQGIIKLGRWNPLPLSYVTDAPDATVADMLQDVYHVVTLKIQLQSCSKLEDLPAEQWNHATVRNALKELLKEMNQSTLAKECPLSQSMISSIVNSTYYANVSATKCQEFGRWYKKYKKIKVERVERENLSDYCVLGQRPMHLPNMNQLASLGKTNEQSPHSQIHHSTPIRNQVPALQPIMSPGLLSPQLSPQLVRQQIAMAHLINQQIAVSRLLAHQHPQAINQQFLNHPPIPRAVKPEPTNSSVEVSPDIYQQVRDELKRASVSQAVFARVAFNRTQGLLSEILRKEEDPRTASQSLLVNLRAMQNFLNLPEVERDRIYQDERERSMNPNVSMVSSASSSPSSSRTPQAKTSTPTTDLPIKVDGANVNITAAIYDEIQQEMKRAKVSQALFAKVAANKSQGWLCELLRWKENPSPENRTLWENLCTIRRFLNLPQHERDVIYEEESRHHHSERMQHVVQLPPEPVQVLHRQQSQPTKESSPPREEAPPPPPPTEDSCAKKPRSRTKISLEALGILQSFIHDVGLYPDQEAIHTLSAQLDLPKHTIIKFFQNQRYHVKHHGKLKEHLGSAVDVAEYKDEELLTESEENDSEEGSEEMYKVEAEEENADKSKAAPAETDQR

SEQ ID NO: 7 - DNA, 5052 bp, Homo sapiens
aagtactgaactcctaatatcaccaattcttctaagttcctggacattgatccggaggaggattcgcagttcaacatcaaggtccctgtgcgttttattgcgacctgccggtgggaactttgtctccgagtcggagcagcatggagcggcggagcgagagcccgtgtctgcgggacagccccgaccggcggagcggcagcccggacgtcaaggggcctcccccagtgaaggtggcccggctggagcagaacggcagccccatgggagcccgcgggaggcccaacggcgccgtggccaaggccgtgggaggtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaagtggaaagagtggaacgagaaaacctttcagactattgtgttctgggccagcgtccaatgcatttaccaaatatgaaccagctggcatccctggggaaaaccaacgaacagtctcctcacagccaaattcaccacagtactccaatccgaaaccaagtgcccgcattacagcccatcatgagccctggtcttctttctccccagcttagtccacaacttgtaaggcaacaaatagccatggcccatctgataaaccaacagattgccgttagccggctcctggctcaccagcatcctcaagccatcaaccagcagttcctgaaccatccacccatccccagagcagttaagccagagccaaccaactcttccgtggaagtctctccagatatctaccagcaagtcagagatgagctgaagagggccagtgtgtcccaagctgtctttgcaagagtggcattcaaccgcacacagggattgttgtctgagattctgcgtaaggaagaagaccctcggacagcctctcagtctcttctagtaaacctgagggccatgcagaatttcctcaatctgccagaagtggagcgagatcgcatctaccaggatgagagggagcggagcatgaatcccaatgtgagcatggtctcctcggcctccagcagtcccagctcctcccgaacccctcaggccaaaacctcgacaccgacaacagacctccctattaaggtggacggcgccaacatcaacatcacagctgccatttatgacgagatccaacaggagatgaaaagggccaaggtgtctcaagccctgtttgccaaagtggctgcaaataaaagtcagggctggctgtgtgaactgctccgctggaaggagaacccaagcccagaaaaccgcaccctctgggaaaacctctgtaccatccgtcgcttcctgaaccttccccagcatgagagggatgtcatctatgaggaggagtcaaggcatcaccacagcgaacgcatgcaacacgtggtccagcttccccctgagccggtgcaggtacttcatagacagcagtctcagccagccaaggagagttcccctcccagagaagaagcgcctcccccacctcctccgactgaagacagttgtgccaaaaagccccggtctcgcacaaagatctccttagaagccctggggatcctccaaagctttattcatgatgtaggcctgtacccagaccaggaagccatccacactctttcggctcagctggatctccccaaacacaccatcatcaagttcttccagaaccagcggtaccacgtgaagcaccacgggaagctgaaagagcacctgggctccgcggtggacgtggctgaatataaggacgaggagctgctgaccgagtcagaggagaacgacagcgaggaaggctccgaggagatgtacaaagtggaggctgaggaggaaaatgctgacaaaagcaaggcagcacctgccgaaattgaccagagataatgtgaacttctactaggcaaagcaatacatcggtccaaggattttctgctttcatttctttaaaagttttttgttagtttgttttttgtttttgtttttgggtttttttggctttatttttgtctttttatgtctgttttgtttttcttacccttttggacatttctttgttgcacaggatacacctatagactgaataagttcagtatttccgaatcagacatcgccttggcaaagacactaaagcgttacactttatcccgtctctatgactggatcatagtcattataatcacaggagactctgccttcattatccttgcacttaacggaagttacatcaggcaagtaccaggatgaaaagaactatgaaataaatgaaggaagctacaagtgtgtgtgtatatgtatatgtatatatctctatatttacatatatatattaaaattgcatgggacagagactttgcaatccgaaagaatagactgtgaaatgagttcttaaagaaaagacttgtttatgtattaaaaaaaccacttcacagtgagtcgctttggctttttgataaactgcggcctgctctcagggtggggtgactatttttgaattcctatttattttttgtgtttgtccctgattttttttttttaattctatggcttcctatctggcagcttaatgggtaatttttgaggtatgtatttaacaaaataaacgacactgccgaaaaaaaaaaaagtgaagtgaaaacaatcagggcacattaaaatgatacaagtcaaataaatcttaaagacacaatgcacacttaaaatgactcaataaaatgacttgctacgttccgttattcaatttgtcattactgtagtgaacagatgcatttctgtggaattccaaataagtaaaactgaaattcagtgcagagaaaactttgtccactagtgcaagtcttgatcaaatgacattttgacattggacatatggaattcatagtatgagccacattttgttgtgaaatttatttacctgcttgtggcttcaaatctgaaaattaataagcctgctcgtttaaaagttgtttgttgttgctgtttttttgtctttttgttttttactagaaaatagttcagtgtaatattaagttagaaaagaagttgctgcccagttaaaggggctccctctcaaataaatctccatccttccctctcccaaaagacatttctgatttctgcttcactttgggcttcctcttcttcgtacacattccatctacctaatcaaacattttcagtccctgatctctcctgtcccttttcctgggatgacagccctaacaagaactgtttttgaatcgttgtgcagctccaggcaatagagtatgtgaagcgatttcagtagaatcacttactcatcctaaaagaaaacattatcccagttacctacatcgcaattaccttatgtaaagcagaactaatgctgactggatgtttaatgggatgagcattaaagctgcaatctactatagtactccagatctctttcggcttcctatgagaaacaccagaagcattactttccacttctacttacagtaattgcaagaggagacctcacattcaggactggcctagtgaacgtaatccatgctttaaactggccattaaacagtcccacatggttggattttttttttttttttgagttgtgctttcacaaaaccttgtcaaagacctcatgcaatatcactttgaaagttattttctgtttactacacaaacattgtaatataactgttaatactatttatatatttgaaaggtataaaaggtaggagttaaaaaaaaaacctctatgtgtagatattaactcagaacttacaatatacagggagaagacatgttgcaatacaagctaattctagctgctcagtaacctctggagtttttaaagggacattttcctgtactttttcaaataatgatgtttaaaaattatcttgacataagcgtcatatacctttgcaaaaggatggttgtttgcagttagccctggccccatccttcctatttctgtagtatgctgcagctttaatcagaaagtccatggttgctgcttcctgatctccgagttactctttccaaattgtcttcttacactgttgctgaaggtcactctgtacacgtaatggaaactgattttgccaagctcttacaaggtggttcatctatcgatggcatccgcatttggtatcttttacacttcaaccaaaaatttattaggtatttttcaatgctaagtcttgccttttattttttaatttcactgccaagtttgcagtggttctaagtgaatctgtgggcattttagcctgtggtcttgccagatctttgcgaattacaatgcatatatgtctatttattcaatatctgtcatataatatctatttggaagaagaaactttctcttgtagtgcctcttgacaaagcacaatttcccgcctttttttttttttgtgaaatgaaaaaaacaaattgtgttttattgcggtatcaacaatgtgaataaggattaacatattgtaaatgttcttttttccatgtaaatcaactatctttgttatcactaagtgataattaatttttaacttatgtgcattgttaggctgttagaattttttggttgttaaaataaacgcattcaataaatatg

SEQ ID NO: 8 - protein, 733 aa, Homo sapiens
MERRSESPCLRDSPDRRSGSPDVKGPPPVKVARLEQNGSPMGARGRPNGAVAKAVGGLMIPVFCVVEQLDGSLEYDNREEHAEFVLVRKDVLFSQLVETALLALGYSHSSAAQAQGIIKLGRWNPLPLSYVTDAPDATVADMLQDVYHVVTLKIQLQSCSKLEDLPAEQWNHATVRNALKELLKEMNQSTLAKECPLSQSMISSIVNSTYYANVSATKCQEFGRWYKKYKKIKVERVERENLSDYCVLGQRPMHLPNMNQLASLGKTNEQSPHSQIHHSTPIRNQVPALQPIMSPGLLSPQLSPQLVRQQIAMAHLINQQIAVSRLLAHQHPQAINQQFLNHPPIPRAVKPEPTNSSVEVSPDIYQQVRDELKRASVSQAVFARVAFNRTQGLLSEILRKEEDPRTASQSLLVNLRAMQNFLNLPEVERDRIYQDERERSMNPNVSMVSSASSSPSSSRTPQAKTSTPTTDLPIKVDGANINITAAIYDEIQQEMKRAKVSQALFAKVAANKSQGWLCELLRWKENPSPENRTLWENLCTIRRFLNLPQHERDVIYEEESRHHHSERMQHVVQLPPEPVQVLHRQQSQPAKESSPPREEAPPPPPPTEDSCAKKPRSRTKISLEALGILQSFIHDVGLYPDQEAIHTLSAQLDLPKHTIIKFFQNQRYHVKHHGKLKEHLGSAVDVAEYKDEELLTESEENDSEEGSEEMYKVEAEEENADKSKAAPAEIDQR

SEQ ID NO: 9 - DNA, 19 bp, Artificial Sequence
  SATB1 forward primer
gggaagagaaaataataca

SEQ ID NO: 10 - DNA, 19 bp, Artificial sequence
  SATB1 reverse primer
tttcctaaggttggttttc

SEQ ID NO: 11 - DNA, 24 bp, Artificial sequence
  SATB2 forward primer
atcatcatcataacaaccatctcc

SEQ ID NO: 12 - DNA, 22 bp, Artificial sequence
  SATB2 reverse primer
gctccagccgggccaccttcac

SEQ ID NO: 13 - DNA, 24 bp, Artificial sequence
  SATB2 forward primer 2
gagatgtacaaagtggaggctgag

SEQ ID NO: 14 - DNA, 24 bp, Artificial sequence
  SATB2 reverse primer 2
ctgtgaagtggtattagtttttat

SEQ ID NO: 15 - DNA, 25 bp, Artificial sequence
  SATB1 control genomic forward primer
cgttggtacttgtaggagctgaagc

SEQ ID NO: 16 - DNA, 24 bp, Artificial sequence
  SATB1 control genomic reverse primer
ggcagagatttggtgaatgtaagc

(19) United States
(12) Patent Application Publication
(10) Pub. No.: US 2011/0230547 A1
(43) Pub. Date: Sep. 22, 2011

(54) Potential Prognostic Markers and Therapeutic Targets for Neurological Disorders

(75) Inventors: Terumi Kohwi-Shigematsu, El Cerrito, CA (US); Yoshinori Kohwi, El Cerrito, CA (US)

(73) Assignee: The Regents of the University of California, Oakland, CA (US)

(21) Appl. No.: 12/970,878

(22) Filed: Dec. 16, 2010

Related U.S. Application Data
(63) Continuation of application No. PCT/US2009/047571, filed on Jun. 16, 2009.
(60) Provisional application No. 61/061,663, filed on Jun. 16, 2008.

(51) Int. Cl. A61K 31/7088; C07H 21/04; A61P 25/00; A01K 67/00; C40B 40/06; C12Q 1/68

(52) U.S. Cl. 514/44 R; 536/23.1; 800/13; 800/18; 800/9; 506/16; 435/6.11

(57) ABSTRACT

We recently found that Satb1 is expressed highly by mature neurons in specific regions of the postnatal brain. Satb2, a homolog of Satb1, is expressed at low levels in the postnatal brain. Neurons respond to external stimuli and rapidly and dynamically change their expression. Satb1 has been found to directly regulate a set of genes in the postnatal brain, presumably playing a crucial role as a ‘genome organizer’ for brain function and behaviors. Satb2 may also have a similar function, even though it is expressed at low levels in the postnatal brain. The present invention describes compositions, reagents and tools using wild type and variant SATB1 and SATB2 genes and proteins for use in diagnosis, prognosis and therapeutics in neurological dysfunction and psychiatric disorders.

### CROSS-REFERENCE TO RELATED APPLICATIONS

This application claims priority to PCT International Application No. PCT/US2009/047571, filed on Jun. 16, 2009, and to U.S. Provisional Patent Application No. 61/061,663, filed on Jun. 16, 2008, both of which are hereby incorporated by reference in their entirety.

This application is also related to co-pending U.S. patent application Ser. No. 12/058,574, which claims priority from PCT International Application No. PCT/US2006/038711, which also claims priority from U.S. Provisional Patent Application No. 60/722,833, all of which are hereby incorporated by reference.

### STATEMENT OF GOVERNMENTAL SUPPORT

This invention was made during work supported under Grant No. R37CA039681-24 awarded by the National Cancer Institute of the National Institutes of Health, and under Contract No. DE-AC02-05CH11231 awarded by the U.S. Department of Energy. The government has certain rights in this invention.

### REFERENCE TO ATTACHED SEQUENCE LISTING AND TABLE APPENDIX

This application incorporates by reference in its entirety, the attached sequence listing found in paper form. This application incorporates by reference in its entirety, the attached table, Table 1, found in paper form.

### BACKGROUND OF THE INVENTION

1. Field of the Invention

The present invention relates to markers and therapeutics for neurological disease. More specifically, the present invention relates to the detection of a general neurological marker which serves as an indicator of neurological disease and or dysfunction.

2. Related Art

Different patterns of expression for a large number of genes have been correlated with cancer development and/or progression. Although different mutation events can ultimately lead to the development of metastatic cancer in different patients, there must be a common and fundamental molecular mechanism allowing carcinoma cells to acquire such an aggressive phenotype and to maintain it. It is likely that such a mechanism exists at the level of DNA organization in cells.

A cell must organize the enormous length of DNA into a tiny space of the cell's nucleus, in order to express only those genes relevant for that cell's function. Our recent work on the protein SATB1 has shed light into the mystery of how this ‘functional’ packaging is accomplished. SATB1 organizes genomic DNA sequences by providing an intra-nuclear architecture, onto which a group of specialized DNA sequences are anchored and assembled, with those various enzymes and protein factors necessary for gene expression. Thus SATB1 acts as a genome organizer and controls numerous genes.

One of the inventors has been studying SATB1 for many years. SATB1 is described in U.S. Pat. No. 5,652,340 and antibodies made thereto are described in U.S. Pat. No. 5,869,621, which are hereby incorporated by reference.

SATB1, which is expressed predominantly in the T cell lineage (Dickinson, L. A., T. Joh, Y. Kohwi, and T. Kohwi-Shigematsu, Cell 70:631, 1992; Alvarez, J. D., Yasui, D. H., Niida, H., Joh, T., Loh, D. Y., and Kohwi-Shigematsu, T., Genes Dev 14, 521-535, 2000), has been shown to provide a unique nuclear architecture onto which chromatin is folded by anchoring specialized DNA sequences (Yasui et al., Nature 419:641-645, 2002; Cai et al., Nat. Genet. 37:31-40, 2003). These specialized DNA sequences are called base unpairing regions (BURs), which are double-stranded DNA highly potentiated for base unpairing under negative superhelical strain (Kohwi-Shigematsu, T. and Kohwi, Y. Biochemistry, 29:9551-9560, 1990: Bode, J., Kohwi, Y., Dickinson, L. Joh, T., Klehr, D., Mielke, C., and Kohwi-Shigematsu, T. Science, 255:195-197, 1992). SATB1 represents a new class of gene regulator: by targeting chromatin remodeling/modifying complexes to the DNA sequences anchored to the SATB1 nuclear architecture, it thus regulates chromatin structure over long distances as well as expression of numerous genes (Yasui, D., Miyano, M., Cai, S., Varga-Weisz, P., and Kohwi-Shigematsu, T. (2002) Nature 419, 641-645; Cai, S., Han, H. J., and Kohwi-Shigematsu, T. (2003) Nat Genet. 34, 42-51).

It was previously shown that SATB1 confers a cage-like nuclear architecture, surrounding heterochromatin, in thymocytes and activated T cells, onto which specific subgroups of gene loci are tethered to form loop configuration (Cai et al., 2003). SATB1 recruits and assembles gene loci with chromatin remodeling enzymes and transcription factors, either to activate genes (e.g. c-myc in thymocytes, Th2 cytokine genes in activated T cells) or to repress them (e.g. IL-2Rα in thymocytes) (Yasui et al., 2002). By recruiting chromatin-modifying enzymes such as HDAC1 or DNA methyltransferases, SATB1 also dictates the epigenetic status of chromatin. When T helper cells are activated, SATB1 is induced to form dense chromatin looping in the 200 kb-cytokine gene cluster region to induce cytokine genes (Cai et al., 2006).

We previously identified SATB1 as a genome organizer in the T cell lineage, acting as a cell-type specific nuclear protein which orchestrates the temporal and spatial expression of numerous genes during T cell differentiation (Alvarez, J. D., Yasui, D. H., Niida, H., Joh, T., Loh, D. Y., and Kohwi-Shigematsu, T. (2000) Genes Dev 14, 521-535). SATB1 is most highly expressed in thymocytes, exhibiting cage-like nuclear distribution surrounding heterochromatin. SATB1 is essential for proper T cell development: in its absence, T cell differentiation is blocked at the CD4CD8 double positive stage, preventing them to mature. SATB1 has a novel role as a global gene regulator, functioning through organizing higher-order chromatin structure by anchoring target genes, through binding to specialized DNA sequences associated with each gene locus, onto the SATB1 regulatory network. SATB1 recruits and assembles these gene loci with chromatin remodeling enzymes and transcription factors, either to activate or repressed genes. Such activity of SATB1 is also required for T cell activation. When T helper cells are activated, SATB1 is induced and brings distant cytokine gene loci into close spatial proximity by binding to the specializing sequences associated with these gene. Transcription complexes are also recruited to the closely juxtaposed gene loci, enabling simultaneous expression of multiple cytokine genes after stimulation.

Most recently, we reported a yet new function of SATB1 in breast cancer metastasis (Han H J, Russo J, Kohwi Y, Kohwi-Shigematsu T., SATB1 reprograms gene expression to promote breast tumour growth and metastasis, Nature. 2008 Mar. 13; 452(7184):187-93; and WO 2007/075206). In breast cancer cells, once SATB1 becomes expressed, it tethers its target genes onto its network and establishes local epigenetic status of chromatin, and changes the expression profiles by affecting transcription of ˜1000 genes to promotes breast cancer metastasis. It has never been expected that SATB1, which has been thought to be cell type-specific and necessary for T cell development, would also be expressed in breast cancer cells, primarily in metastatic breast cancer cells. SATB1 also acts as a genome organizer in metastatic breast cancer and regulates key players necessary for the metastatic activity of breast cancer.

SATB1 plays a crucial role as a genome organizer in many tissues. Therefore, if SATB1 can be found to be expressed in the brain, it is expected to play a role in regulating expression of a large number of genes. It has not yet been shown whether SATB1 is such a critical factor for brain function. There has been no report about genome organizer function in brain function.

The human genome contains about 25,000 genes, expression of which is tightly regulated in a cell-type and developmental stage-specific manner. When neurons receive stimuli, rapid and specific changes in gene expression takes place. Such dynamic change in gene expression is essential for neuronal function such as memory. To facilitate such changes in gene expression, we expect that proteins that organize chromatin structure and gene expression (genome organizers) play a pivotal role. A genome organizer would efficiently “guide” transcription factors and chromatin remodeling enzymes to find their target genes.

Neurons are different from the majority of terminally differentiated cells because gene expression in neurons is highly varied, with a large proportion of the coding genome being expressed at any time point (Geschwind 2000, Sanndberg 2000). This is most likely explained by activity-regulated transcription in the CNS, which is believed to regulate many aspects of neuronal functions, e.g. neuronal differentiation, survival, synapse formation, synaptic plasticity, and memory consolidation. It is known that synaptic activity induced by stimuli leads to activation of different signaling cascades. In response to such signaling, the transcriptional regulator cAMP-responsive element binding protein (CREB) plays a crucial role in associating synaptic activity with long-term changes in the CNS. CREB-dependent gene expression has been associated with memory consolidation, addiction, circadian rhythmicity, and developmental plasticity.

Although the key role of CREB in gene expression in the CNS is well established, it is still poorly understood how different subsets of CREB target genes are selectively activated in neurons depending on the type of stimulus and how genes that are independent of CREB could be activated or repressed in these neurons.

However, at each individual transcription factor level, such as CBP, there has been a large body of literature description transcription factor function and if there is a connection to neurological disease. Early behavioral studies of learning and long-term memory revealed a requirement for both new protein synthesis and gene transcription (reviewed in Kandel, Science 294:1030-1038, 2001). CREB is a transcription factor that is activated through phosphorylation in response to a vast array of physiological stimuli. CREB is considered to be an important factor in learning and memory because CREB-dependent gene expression contributes critically to long-term memory and plasticity in vertebrates.

It is not known, however, how different stimuli activate different subsets of CREB target genes and other transcription factor target genes. Neurons are unique among terminally differentiated cells in that they are constantly subjected to dynamic changes in gene expression in response to stimuli, However, it is not known whether neurons must also use a mechanism, involving a genome organizer like SATB1, to regulate expression of a large body of genes. There has been no report on higher-order chromatin structure and brain function, except for a study on a defect in chromatin looping in Rett syndrome, a neurodevelopmental disorder (Horike et al., Loss of silent-chromatin looping and impaired imprinting of DLX5 in Rett syndrome, Nat. Genet. 2005 January; 37(1):31-40. Epub 2004 Dec. 19).

Others have identified SATB2, a SATB1 homolog, in brain tissues in rats and as a cleft palate gene. See Szemes M, Gyorgy A, Paweletz C, Dobi A, Agoston D V., Isolation and characterization of SATB2, a novel AT-rich DNA binding protein expressed in development- and cell-specific manner in the rat brain, Neurochem Res. 2006 February; 31(2):237-46. Epub 2006 Apr. 4; FitzPatrick D R, et. al., Identification of SATB2 as the cleft palate gene on 2q32-q33, Hum Mol. Genet. 2003 Oct. 1; 12(19):2491-501. Epub 2003 Jul. 29.

### SUMMARY OF THE INVENTION

We previously identified SATB1 as a genome organizer in the T cell lineage, and as a prognostic marker for metastatic cancer. In the postnatal mouse brain, we recently found Satb1 expressed at high levels, while its homolog Satb2 expressed at low levels, by mature neurons in the postnatal cortex and a specific subset of neurons in hippocampus. Satb1, but not Satb2, is expressed in the amygdala. Like activated T cells, neurons respond to external stimuli and rapidly and dynamically change their expression. Thus, it may be that the SATB1 and/or SATB2 are utilized as an ‘organizer’ as well for brain function, such as learning and memory.

The present invention involves the SATB1 and/or SATB2 gene and their expressed products, SATB1 and SATB2 protein, and a newly found association with neurological dysfunction that may lead to psychiatric disorders.

The invention provides non-human animals in which Satb1 exons are flanked by loxp sites in one of the alleles (Satb1 floxed mice) and non-human animals with knockout expression of Satb1 specifically in cells expressing synapsin, which correspond to mature neurons. Thus, in one embodiment, a non-human animal comprising a genome which is Satb1^{flox/flox} homozygous or Satb1^{flox/+} heterozygous.

The invention further provides a conditional homozygous Satb1-null non-human animal comprising a genome having a functionally disrupted Satb1 gene, wherein the Satb1 gene is disrupted only in neurons in a developmental stage specific manner or brain subregion-specific manner. In a preferred embodiment, the animal is a mouse and is a Satb1^{flox/flox}: Synapsin promoter-driven-Cre and/or a Satb1^{flox/flox}: CamKII promoter-driven-Cre recombinase mouse. These animals in which Satb1 is depleted from mature neurons have increased aggression, greatly decreased anxiety, hyperactivity, impaired learning and memory, and display other abnormal physical, behavioral and psychological behavior, including seizures. This invention also includes DNA conditional knockout-targeting constructs, such as the one used by the inventors to delete mouse Satb1 and was built using PCR products and primers made from SEQ ID NO: 1.

The animal models also may find use in studies to confirm association and causative effect of Satb1 depletion on neurological dysfunction or disease.

This invention also provides non-human animals for further animal studies by pharmaceutical companies to study SATB1 and SATB2 function in mature neurons in postnatal brain or in any other cell types. Depletion of Satb1 in any cell type can be achieved by cross-breeding with transgenic mice containing Cre-recombinase driven by cell-type specific promoters. The invention is useful for animal studies that explore the regulation and expression of target genes of mouse Satb1 in mature neurons or any other cell type of interest, Satb1 protein interaction with other proteins, and further in vivo study of Satb1 and SATB2.

It is expected that various polymorphisms will be identified which are correlated with different SATB1 and/or SATB2 levels of expression, function of these proteins, and/or different phenotypic responses. Specifically, individuals having SNPs in the regulatory regions or in the SATB1 or SATB2 coding regions will likely display some neurological dysfunction and/or psychiatric disorder.

Thus, the invention includes using various methods for screening for genetic SATB1 and/or SATB2 genotypes or SNPs in humans. In one aspect, the invention also provides a synthetic polynucleotide having a sequence encompassing a single nucleotide polymorsphism (SNP) or variant sequence in SEQ ID NO: 3 and/or SEQ ID NO:7 for use in detecting the presence of a wild-type or a rare allele of said SNPs, wherein the presence of the SNP is associated with a neurological disorder or disease.

Other methods for diagnostic purposes in this invention include but are not limited to, making antibodies to SATB1 and/or SATB2 variants, attachment of the SATB1 and/or SATB2 variant sequences disclosed herein onto solid supports for array and gene chips, and other hybridization assays and amplifying or sequencing SATB1 and/or SATB2 for genotyping.

In another aspect, an array of oligonucleotide probes to detect a single nucleotide polymorphism in variant SATB1 and/or SATB2 in patient sample, comprising at least one oligonucleotide representing wild type SATB1 and/or SATB2 and one oligonucleotide representing variant SATB1 and/or SATB2, wherein the detection of a variant SATB1 and/or SATB2 indicates the patient may have an associated neurological disfunction or disease. In one embodiment, the array comprising oligonucleotide probes representing all publicly available SATB1 and SATB2 SNPs and novel SATB1 and SATB2 SNPs found, thereby providing an array platform for diagnostic detection.

It is an object of the invention to provide a method for determining the genetic status of an individual, comprising: detecting the presence of one of more single nucleotide polymorphisms (SNPs) in SATB1 or SATB2 in the DNA or RNA of the individual; and whereby the presence of one or more SNPs indicates neurological dysfunction in the individual.

In another aspect of the invention, an antibody to variant SATB1 peptide encoded by a SATB1 variant oligonucleotide containing a SNP or an antibody to variant SATB2 peptide encoded by a SATB2 oligonucleotide containing a SNP.

In another embodiment, the present invention provides for a therapeutic composition for restoring impaired SATB1 protein and function in the brain. In one embodiment, the composition is an isolated polynucleotide having at least 70% homology to the cDNA SATB1 SATB2 sequence selected from the group consisting of SEQ ID NO: 3 and SEQ ID NO: 7. In another embodiment, the composition further comprising a vector for gene therapy for delivery to the brain of a subject.

The present invention also provides gene data which shows that SATB1 regulates expression of immediate early genes, neurotransmitter genes, calcium responsible genes, genes related to growth in neurons. Thus, in another aspect, the invention further provides a set of genes directly regulated by SATB1 and/or SATB2 in the brain.

Finally, it is another object of the invention to use of Satb1 or SATB2 to identify crucial genes which, when mutated, lead to neurological disorders, wherein said identification of genes is based on (a) chromatin immunoprecipitation of genes where Satb1 or SATB2 binds in vivo, and (b) expression profiles that show dependency on Satb1 or SATB2.

### BRIEF DESCRIPTION OF THE DRAWINGS

FIG. 1 shows cartoons of activation-dependent looping events and a model of transcriptionally active chromatin. (A) Summary of 3C and ChIP-loop assays of the cytokine regions in resting and activated T helper cells. Black line: converged sites by looping in resting cells. Gray lines above gene sequence line: much increased frequencies for convergence after activation. Gray lines below gene sequence line: sites newly brought into convergence upon activation. (B) A schematic model based on the looping events showing all small loops converging to a common core base bound to the SATB1 network (balls), resulting in reduction of the total physical volume of the active transcriptional complex, presumably enhancing the accessibility of factors to genomic sites. Black vertical arrowheads show direct SATB1-binding sites. (FIG. 3 in pioneer)

FIG. 2. Double in situ hybridization for Satb1 and/or Satb2 on 2-week old mouse brains. In the amygdala (indicated by a while arrow), only Satb1 is transcribed. In the hippocampus and the frontal cortex, both Satb1 and/or Satb2 are transcribed. Some neurons appear to co-express Satb1 and/or Satb2, however, others express only one of the two. Satb1 and/or Satb2 are highly homologous both at their amino acid and DNA sequence levels. Using our SATB1 knockout mice (which die at 2.5-3 weeks postnatally) as a reference, we could optimize the conditions for in situ hybridization to distinguish the two transcripts.

FIG. 3. Satb1 and/or Satb2 are expressed in discrete regions in postnatal mouse brain. Immunostaining of P13 brain coronal sections with Satb1 (A-C) or Satb2 (D-F) specific antibodies countersatined with DAPI is shown. Staining on cross section of wild-type brain's one entire hemisphere for Satb1 (A) or Satb2 (D) is shown. Boxed area of hippocampus is magnified in lower panels for Satb1 (B) and Satb2 (E). Similar area from Satb1−/− brain stained for Satb1 (C) or Satb2 (F) is presented. Amygdala, exclusively expressing only Satb1 protein is indicated with arrows. (G-N) mRNA fluorescent in situ hybridization of P13 wild-type coronal sections with Stab 1 (G) and Satb2 (H) specific antisense riboprobes and merged image of these stainings (I) is shown. Boxed area on I is magnified in lower panel (J-L). Similar area from Satb1−/− sections stained with Satb1 (M) or Satb2 (N) antisense riboprobes is used to show the specificity of the staining Scale bar, 500 μm.

FIG. 4. Satb1 and/or Satb2 expressing cells in postnatal brain are postmitotic neurons. Double immunostaining of SATB family proteins with neuronal marker NeuN (A-I) or astrocyte marker Gfap (J-R) is shown. Counterstaining with DAPI is used to visualize dentate gyms in hippocampus. All cells expressing Satb family proteins are also NeuN-positive in all studied areas, including cortex (A-C), dentate gyms (D-F) and lateral nucleus of amygdala (G-I). No colocalization of SATB family proteins with Gfap was detected in cortex (J-L), dentate gyms (M-O) nor in amygdala (P—R).

FIG. 5. The expression of Satb1 and/or Satb2 is age-dependent. (A) Relative transcript levels of Satb1 and/or Satb2 in cortex at different ages (P0 to 6 weeks) were measured by qRT-PCR. Values for each time point represent means of three biological replicates. (B) Satb1 and/or Satb2 protein levels in cortex extracts from mice in different ages were determined by Western blot by using anti-SATB1 mouse monoclonal (reacts with both SATB proteins), purified anti-SATB1 rabbit polyclonal or anti-SATB2 polyclonal antibody. Anti-Tubulin antibody was used as loading control.

FIG. 6. Genes involved in different aspects of brain function are dysregulated in Satb1-null cortex. (A) List of representative genes found by microarray analysis to be significantly (p-value <0.05) dysregulated in P13 Satb1-null cortex compared to wild-type cortex. Genes selected for subsequent testing by quantitative RT-PCR are indicated in bold. (B) Functional clustering based on GO annotation of genes significantly up or down regulated more than 1.3 folds in Satb1-null cortex compared to wild-type cortex. (C) Quantitative RT-PCR analysis to confirm microarray analysis results. Transcript levels relative to that of Actin-β for 7 genes significantly dysregulated based on microarray and 3 additional immediate early genes (Egr1, Fos and Arc) in wild-type (dark blue) and Satb1-null (purple) cortex. Values for each gene in knockout sample in each separate litter were normalized to the wild-type value in that litter. Values represent means±standard deviation of three or four experimental replicates for each genotype.

FIG. 7. SATB family proteins bind immediate early genes in vivo and thus regulate their expression directly. Chromatin immunoprecipitation was performed with either anti-SATB antibody or preimmune serum on Sau3A-digested chromatin from one-day-old wild-type brain. PCR amplification results from Satb-ChIP samples, preimmune-ChIP samples and input DNA are shown for Arc (A), Fos (B), Egr1 (C) and Egr2 (D). Exons on each genomic sequence are marked with green boxes, CpG islands with pink boxes and translation initiation sites by black arrows. Sau3AI digestion sites are indicated. The positions on genomic sequence where primers are designed are shown by numbers. Numbers indicating genomic sites bound by Satb family proteins are in red.

FIG. 8. Satb1 regulates activity-dependent gene expression. Organotypic cortical slice cultures prepared from Satb1 knockout or wild-type brains were treated with 25 μM forskolin for different amount of time (1 h, 2 h and 4 h). 0.1% DMSO treated samples were used as a control (0 h). qRT-PCR was used to determine the expression levels of 84 different cAMP or Ca2+ responsive genes in wild type and knockout cultures. Transcript levels for each gene were normalized to the average of combination of three internal control genes (Hprt, Gusb and Hsp90ab1). Fold induction relative to 0 h at different time points in wild-type (blue) and knockout (purple) is presented. Values for each data point represent means±standard error of three or four experimental replicates. *P<0.05, **P<0.005, Student's t test relative to wild-type.

FIG. 9 shows targeting constructs and breeding schemes for SATB1 conditional knockout animals.

FIG. 10 is a graph of amount of time conditional knockout (CKO) animals spent in each section of the elevated plus maze as compared to control animals.

FIG. 11 is a graph of the number of injured animals in conditional knockout animal cages versus control cages.

FIG. 12 is a graph of the amount of time conditional knockout (CKO) animals spent with a novel or old object as compared to the amount of time control animals spent.

FIG. 13 is a graph showing the amount of activity of conditional knockout (CKO) as compared to control animals over a period of time.

FIG. 14A is a graph comparing grip strength of hind limbs of conditional knockout (CKO) as compared to control animals and FIG. 14B is a graph comparing coordination and balance on a rotarod of conditional knockout (CKO) as compared to control animals.

Table 1 shows SATB1-dependant altered genes within 81 cAMP and Ca2+ responsive genes in 13 days cerebral cortex.

### DETAILED DESCRIPTION OF THE PREFERRED EMBODIMENT

### Abbreviations

SATB1; Special AT-rich binding protein 1
HMEC; Human mammary epithelial cell lines
SATB1-siRNA or SATB1-shRNA; Short hairpin-interfering RNAs against SATB1
2D culture; Two dimensional culture, Plastic dish culture
3D culture; Three dimensional culture on matrigel
RT-PCR; Reverse Transcription-Polymerase chain reaction
GO; Gene ontology
EMT; Epithelial-mesenchymal transition
ChIP; Chromatin immunoprecipitation
LM-PCR; Ligation mediated polymerase chain reaction
IL-2Rα; Interleukin-2 receptor alphaα
BRMS1; Breast carcinoma metastasis suppressor 1
PLAUR; Plasminogen activator urokinase
OB; Osteoblast
BM; basement membrane

### DEFINITIONS

“SATB1 and/or SATB2,” as used herein, refers to the amino acid sequences of substantially purified SATB1 and/or SATB2 obtained from any species, particularly a mammalian species, including bovine, ovine, porcine, murine, equine, and preferably the human species, from any source, whether natural, synthetic, semi-synthetic, or recombinant.

The term “agonist,” as used herein, refers to a molecule which, when bound to SATB1 and/or SATB2, increases or prolongs the duration of the effect of SATB1 and/or SATB2. Agonists may include proteins, nucleic acids, carbohydrates, or any other molecules which bind to and modulate the effect of SATB1 and/or SATB2.

An “allelic variant,” as this term is used herein, is an alternative form of the gene encoding SATB1 and/or SATB2. Allelic variants may result from at least one mutation in the nucleic acid sequence and may result in altered mRNAs or in polypeptides whose structure or function may or may not be altered. Any given natural or recombinant gene may have none, one, or many allelic forms. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding SATB1 and/or SATB2, as described herein, include those sequences with deletions, insertions, or substitutions of different nucleotides, resulting in a polynucleotide the same as SATB1 and/or SATB2 or a polypeptide with at least one functional characteristic of SATB1 and/or SATB2. Included within this definition are polymorphisms which may or may not be readily detectable using a particular oligonucleotide probe of the polynucleotide encoding SATB1 and/or SATB2, and improper or unexpected hybridization to allelic variants, with a locus other than the normal chromosomal locus for the polynucleotide sequence encoding SATB1 and/or SATB2. The encoded protein may also be “altered,” and may contain deletions, insertions, or substitutions of amino acid residues which produce a silent change and result in a functionally equivalent SATB1 and/or SATB2. Deliberate amino acid substitutions may be made on the basis of similarity in polarity, charge, solubility, hydrophobicity, hydrophilicity, and/or the amphipathic nature of the residues, as long as the biological or immunological activity of SATB1 and/or SATB2 is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

The terms “amino acid” or “amino acid sequence,” as used herein, refer to an oligopeptide, peptide, polypeptide, or protein sequence, or a fragment of any of these, and to naturally occurring or synthetic molecules. In this context, “fragments,” “immunogenic fragments,” or “antigenic fragments” refer to fragments of SATB1 and/or SATB2 which are preferably about 5 to about 15 amino acids in length, most preferably 14 amino acids, and which retain some biological activity or immunological activity of SATB1 and/or SATB2. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

“Amplification,” as used herein, relates to the production of additional copies of a nucleic acid sequence. Amplification is generally carried out using polymerase chain reaction (PCR) technologies well known in the art. (See, e.g., Dieffenbach, C. W. and G. S. Dveksler (1995) PCR Primer, a Laboratory Manual, Cold Spring Harbor Press, Plainview, N.Y., pp. 1-5.)

The term “antagonist,” as it is used herein, refers to a molecule which, when bound to SATB1 and/or SATB2, decreases the amount or the duration of the effect of the biological or immunological activity of SATB1 and/or SATB2. Antagonists may include proteins, nucleic acids, carbohydrates, antibodies, or any other molecules which decrease the effect of SATB1 and/or SATB2.

As used herein, the term “antibody” refers to intact molecules as well as to fragments thereof, such as Fab, F(ab′).sub.2, and Fv fragments, which are capable of binding the epitopic determinant. Antibodies that bind SATB1 and/or SATB2 polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant,” as used herein, refers to that fragment of a molecule (i.e., an epitope) that makes contact with a particular antibody. When a protein or a fragment of a protein is used to immunize a host animal, numerous regions of the protein may induce the production of antibodies which bind specifically to antigenic determinants (given regions or three-dimensional structures on the protein). An antigenic determinant may compete with the intact antigen (i.e., the immunogen used to elicit the immune response) for binding to an antibody.

The term “antisense,” as used herein, refers to any composition containing a nucleic acid sequence which is complementary to the “sense” strand of a specific nucleic acid sequence. Antisense molecules may be produced by any method including synthesis or transcription. Once introduced into a cell, the complementary nucleotides combine with natural sequences produced by the cell to form duplexes and to block either transcription or translation. The designation “negative” can refer to the antisense strand, and the designation “positive” can refer to the sense strand.

As used herein, the term “biologically active,” refers to a protein having structural, regulatory, or biochemical functions of a naturally occurring molecule. Likewise, “immunologically active” refers to the capability of the natural, recombinant, or synthetic SATB1 and/or SATB2, or of any oligopeptide thereof, to induce a specific immune response in appropriate animals or cells and to bind with specific antibodies.

The terms “complementary” or “complementarity,” as used herein, refer to the natural binding of polynucleotides by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acids strands, and in the design and use of peptide nucleic acid (PNA) molecules.

A “composition comprising a given polynucleotide sequence” or a “composition comprising a given amino acid sequence,” as these terms are used herein, refer broadly to any composition containing the given polynucleotide or amino acid sequence. The composition may comprise a dry formulation, an aqueous solution, or a sterile composition. Compositions comprising polynucleotide sequences encoding SATB1 and/or SATB2 or fragments of SATB1 and/or SATB2 may be employed as hybridization probes. The probes may be stored in freeze-dried form and may be associated with a stabilizing agent such as a carbohydrate. In hybridizations, the probe may be deployed in an aqueous solution containing salts, e.g., NaCl, detergents, e.g., sodium dodecyl sulfate (SDS), and other components, e.g., Denhardt's solution, dry milk, salmon sperm DNA, etc.

“Consensus sequence,” as used herein, refers to a nucleic acid sequence which has been resequenced to resolve uncalled bases, extended using XL-PCR (Perkin Elmer, Norwalk, Conn.) in the 5′ and/or the 3′ direction, and resequenced, or which has been assembled from the overlapping sequences of more than one Incyte Clone using a computer program for fragment assembly, such as the GELVIEW fragment assembly system (GCG, Madison, Wis.). Some sequences have been both extended and assembled to produce the consensus sequence.

As used herein, the term “correlates with expression of a polynucleotide” indicates that the detection of the presence of nucleic acids, the same or related to a nucleic acid sequence encoding SATB1 and/or SATB2, by Northern analysis is indicative of the presence of nucleic acids encoding SATB1 and/or SATB2 in a sample, and thereby correlates with expression of the transcript from the polynucleotide encoding SATB1 and/or SATB2.

A “deletion,” as the term is used herein, refers to a change in the amino acid or nucleotide sequence that results in the absence of one or more amino acid residues or nucleotides.

The term “derivative,” as used herein, refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “similarity,” as used herein, refers to a degree of complementarity. There may be partial similarity or complete similarity. The word “identity” may substitute for the word “similarity.” A partially complementary sequence that at least partially inhibits an identical sequence from hybridizing to a target nucleic acid is referred to as “substantially similar.” The inhibition of hybridization of the completely complementary sequence to the target sequence may be examined using a hybridization assay (Southern or Northern blot, solution hybridization, and the like) under conditions of reduced stringency. A substantially similar sequence or hybridization probe will compete for and inhibit the binding of a completely similar (identical) sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% similarity or identity). In the absence of non-specific binding, the substantially similar sequence or probe will not hybridize to the second non-complementary target sequence.

The phrases “percent identity” or “% identity” refer to the percentage of sequence similarity found in a comparison of two or more amino acid or nucleic acid sequences. Percent identity can be determined electronically, e.g., by using the MEGALIGN program (DNASTAR, Inc., Madison Wis.). The MEGALIGN program can create alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183:626-645.) Identity between sequences can also be determined by other methods known in the art, e.g., by varying hybridization conditions.

“Human artificial chromosomes” (HACs), as described herein, are linear microchromosomes which may contain DNA sequences of about 6 kb to 10 Mb in size, and which contain all of the elements required for stable mitotic chromosome segregation and maintenance. (See, e.g., Harrington, J. J. et al. (1997) Nat. Genet. 15:345-355.)

The term “humanized antibody,” as used herein, refers to antibody molecules in which the amino acid sequence in the non-antigen binding regions has been altered so that the antibody more closely resembles a human antibody, and still retains its original binding ability.

“Hybridization,” as the term is used herein, refers to any process by which a strand of nucleic acid binds with a complementary strand through base pairing.

As used herein, the term “hybridization complex” refers to a complex formed between two nucleic acid sequences by virtue of the formation of hydrogen bonds between complementary bases. A hybridization complex may be formed in solution (e.g., Cot or Rot analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” or “addition,” when referring to a genetic sequence as used herein, refer to changes in an amino acid or nucleotide sequence resulting in the addition of one or more amino acid residues or nucleotides, respectively, to the sequence found in the naturally occurring molecule.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

The term “microarray,” as used herein, refers to an arrangement of distinct polynucleotides arrayed on a substrate, e.g., paper, nylon or any other type of membrane, filter, chip, glass slide, or any other suitable solid support.

The terms “element” or “array element” as used herein in a microarray context, refer to hybridizable polynucleotides arranged on the surface of a substrate.

The term “modulate,” as it appears herein, refers to a change in the activity of SATB1 and/or SATB2. For example, modulation may cause an increase or a decrease in protein activity, binding characteristics, or any other biological, functional, or immunological properties of SATB1 and/or SATB2.

The phrases “nucleic acid” or “nucleic acid sequence,” as used herein, refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material. In this context, “fragments” refers to those nucleic acid sequences which, when translated, would produce polypeptides retaining some functional characteristic, e.g., antigenicity, or structural domain characteristic, e.g., ATP-binding site, of the full-length polypeptide.

The terms “operably associated” or “operably linked,” as used herein, refer to functionally related nucleic acid sequences. A promoter is operably associated or operably linked with a coding sequence if the promoter controls the translation of the encoded polypeptide. While operably associated or operably linked nucleic acid sequences can be contiguous and in the same reading frame, certain genetic elements, e.g., repressor genes, are not contiguously linked to the sequence encoding the polypeptide but still bind to operator sequences that control expression of the polypeptide.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimer,” “primer,” “oligomer,” and “probe,” as these terms are commonly defined in the art.

“Peptide nucleic acid” (PNA), as used herein, refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell. (See, e.g., Nielsen, P. E. et al. (1993) Anticancer Drug Des. 8:53-63.)

The term “sample,” as used herein, is used in its broadest sense. A biological sample suspected of containing nucleic acids encoding SATB1 AND/OR SATB2, or fragments thereof, or SATB1 and/or SATB2 itself, may comprise a bodily fluid; an extract from a cell, chromosome, organelle, or membrane isolated from a cell; a cell; genomic DNA, RNA, or cDNA, in solution or bound to a solid support; a tissue; a tissue print; etc.

As used herein, the terms “specific binding” or “specifically binding” refer to that interaction between a protein or peptide and an agonist, an antibody, or an antagonist. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

As used herein, the term “stringent conditions” refers to conditions which permit hybridization between polynucleotides and the claimed polynucleotides. Stringent conditions can be defined by salt concentration, the concentration of organic solvent (e.g., formamide), temperature, and other conditions well known in the art. In particular, stringency can be increased by reducing the concentration of salt, increasing the concentration of formamide, or raising the hybridization temperature.

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30.degree. C., more preferably of at least about 37.degree. C., and most preferably of at least about 42.degree. C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30.degree. C. in 750 mM NaCl, 75 mM trisodium citrate, and 1% SDS. In a more preferred embodiment, hybridization will occur at 37.degree. C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42.degree. C. in 250 mM NaCl, 25 mM trisodium citrate, 1% SDS, 50% formamide, and 200.mu.g/ml ssDNA. Useful variations on these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25.degree. C., more preferably of at least about 42.degree. C., and most preferably of at least about 68.degree. C. In a preferred embodiment, wash steps will occur at 25.degree. C in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a most preferred embodiment, wash steps will occur at 68.degree. C. in 15 mM NaCl, 1.5 mM trisodium citrate, and 0.1% SDS. Additional variations on these conditions will be readily apparent to those skilled in the art.

The term “substantially purified,” as used herein, refers to nucleic acid or amino acid sequences that are removed from their natural environment and are isolated or separated, and are at least about 60% free, preferably about 75% free, and most preferably about 90% free from other components with which they are naturally associated.

A “substitution,” as used herein, refers to the replacement of one or more amino acids or nucleotides by different amino acids or nucleotides, respectively.

“Transformation,” as defined herein, describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

A “variant” of SATB1 and/or SATB2, as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE™ software.

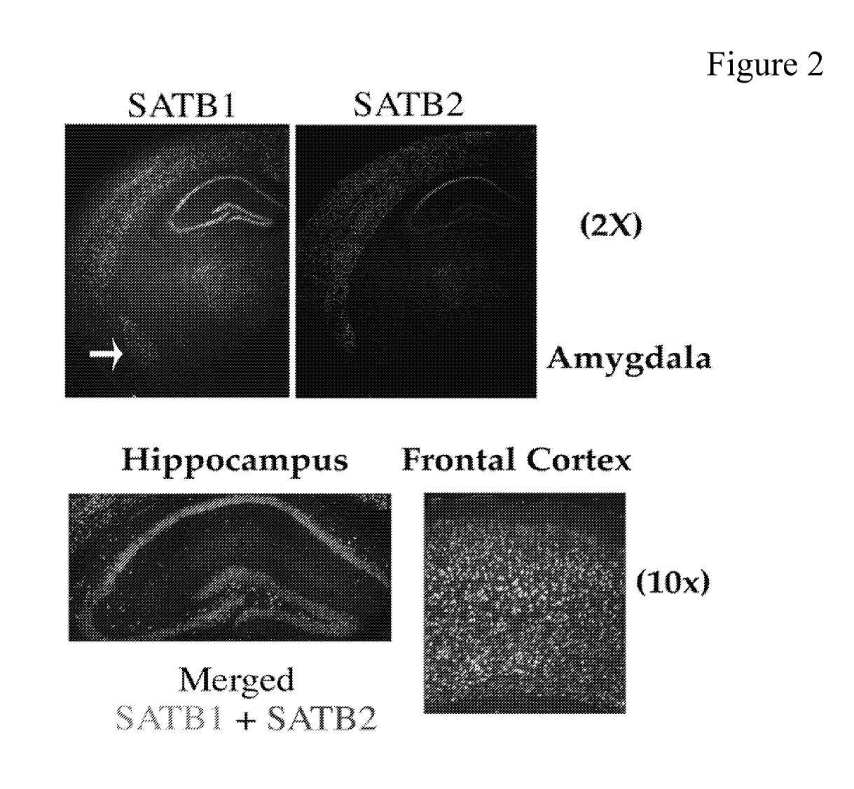

The present invention provides methods and compositions for the diagnosis, and possible treatment, or prevention of neurodegenerative disorders based upon recent discovery by the inventors that SATB1 has a novel role as a global gene regulator, functioning through organizing higher-order chromatin structure in the brain. Although SATB1 was studied almost exclusively for its role in T-cell development and activation, we have recently found SATB1 highly expressed in mature neurons of the postnatal brain. In addition, its close homolog and the only other member known for the SATB family, SATB2, is expressed in mouse embryonic brain. Due to the high homology, it is highly likely that SATB2 has a similar function to SATB1. However, so far no function of SATB2 in the adult brain has yet been studied. By double in situ hybridization, we determined expression of SATB1 and/or SATB2 in the adult mouse brain and found that SATB1 and/or SATB2 are expressed in neurons in multiple subregions of the brain important for memory (hippocampus, frontal cortex, amygdala) (FIG. 2).

It was hypothesized that the SATB family may be important in brain function when unusual behaviors of SATB1 knockout mice were observed. SATB1 straight knockout mice often exhibit unusually high response to outside stimuli and remain mostly isolated from littermates. The initiated studies of these knockout mice showed that SATB1, which functions as a genome organizer in thymocytes and metastatic breast cancer cells, is expressed in specific regions of mouse brain. We also found SATB1 directly regulates many key genes known to play important role in neuronal function and mice deficient in SATB1 from their brain display abnormal behaviors. SATB1 is likely to have a major impact on understanding neuronal function/dysfunction, in disorders or diseases including but not limited to, autism spectrum disorders, attention-deficit disorders, depression, mental retardation, epilepsy, Alzheimer's disease, Parkinson's disease and psychiatric disorders and perhaps give important clues to developing therapies for them.

SATB1 and/or SATB2, a close homolog of SATB1, are expressed in the embryonic brain, and their expression start at early stages during brain development, at E13.5 and E11.5, respectively. In developing cerebral cortex, SATB2 regulates callosal projection neuron identity by repressing Ctip2, a gene required for the formation of corticospinal tract. SATB2 specifically interacts with Ctip2 promoter upstream area and recruits the chromatin remodeling factors histone deacetylase 1 (HDAC1) and metastasis associated protein 2 (MTA2) to this site, suggesting that SATB2 may also have the similar gene regulatory activity like SATB1. Here, we studied SATB family proteins in postnatal mouse brains and found that SATB1 becomes the major protein (among the two SATB family proteins) expressed in postnatal mature neurons. SATB1 is expressed in the amygdala, hippocampus and frontal cortex of the brain, and SATB2 in hippocampus and frontal cortex. We employed a brain culture assay to study activity-dependent expression of a large number of genes depending on SATB1. We found that SATB1 regulates activity-dependent expression of a number of key gene groups in neuronal functions, including CRE-containing genes such as immediate early genes and neurotransmitters.

Among genes which SATB1 regulates, there are several examples of genes extremely important in various brain functions. For example, immediate early gene (IEG) expression is dynamically regulated in brain neurons, in response to natural activity, and linked to neural plasticity. (Li L, Carter J, Gao X, Whitehead J, Tourtellotte W G, The neuroplasticity-associated arc gene is a direct transcriptional target of early growth response (Egr) transcription factors, Mol Cell Biol. 2005 December; 25(23):10286-300). Others have shown that dysfunction of Arc or Egr1 affects the learning and memory processes, ablation of Egr3 expression has an effect on aggressive behavior, on response to stress and novel environment, and on habituation to social clues, all needed for normal social human behavior. Fos had been implicated in regulating sleep, neuronal excitability and also neuronal survival (Merchant-Nancy H, Vázquez J, Aguilar-Roblero R, Drucker-Colín R, c-fos proto-oncogene changes in relation to REM sleep duration, Brain Res. 1992 May 8; 579(2):342-6; Merchant-Nancy H, Vázquez J, García F, Drucker-Colín R, Brain distribution of c-fos expression as a result of prolonged rapid eye movement (REM) sleep period duration, Brain Res. 1995 May 29; 681(1-2):15-22). It has been experimentally shown by others that IEG expression is induced in several neurological disorders. Spreading depression, which has been implicated in several neurological diseases including migraine and stroke, induces the expression of immediate early genes, probably necessary for tissue remodeling and cortical plasticity. A rapid and long-term activation of effector immediate early genes, including Arc, in distinct brain areas is induced following ischemic brain injury (Rickhag M, Teilum M, Wieloch T, Rapid and long-term induction of effector immediate early genes (BDNF, Neuritin and Arc) in peri-infarct cortex and dentate gyms after ischemic injury in rat brain., Brain Res. 2007 Jun. 2; 1151:203-10. Epub 2007 Mar. 12). It has been shown that IEGs Egr1, Egr2 and Fos are induced in human epileptic neocortex (Rakhade S N, Shah A K, Agarwal R, Yao B, Asano E, Loeb J A, Activity-dependent gene expression correlates with interictal spiking in human neocortical epilepsy, Epilepsia. 2007; 48 Suppl 5:86-95, Epilepsia. 2007 December; 48(12):2380). In SATB1-null brain, the induction of several IEG (Egr2 and ATF3) is elevated, indicating that SATB1 functions to modulate the induction of IEGs and SATB1 deficient mice are more prone to epilepsy and ischemic brain injuries.

Aging-induced downregulation of somatostatin (Sst) expression may be a trigger for amyloid β peptide accumulation leading to late-onset sporadic Alzheimer disease. We found that the expression of Sst is downregulated and the induction of expression of Sst receptor 2 (Sstr2) is not properly induced in SATB1-null cortex, suggesting that SATB1 has an effect in modulating the onset of Alzheimer's disease.

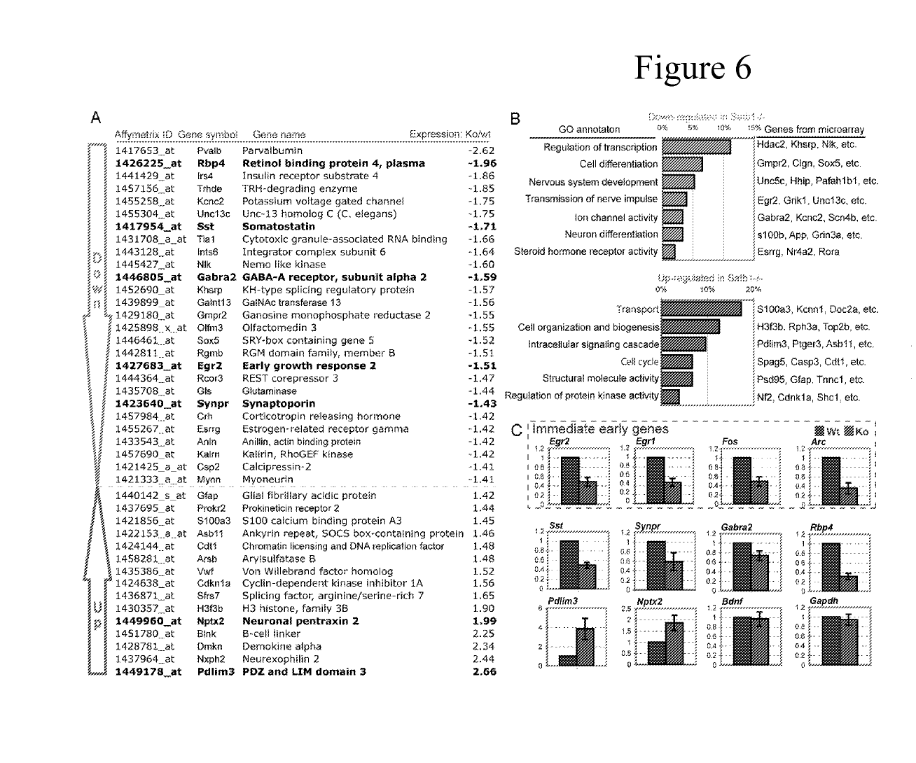

Thus, in one embodiment, the invention provides a set of genes regulated by SATB1 and/or SATB2 in the brain comprising the genes found in Table 1, FIGS. 6, 7 and 8: including FOSB, EGR2, FOS, CYR61, TH, ATF3, AREG, PRL, EGR1, SST, GCG, PLF, S100A8, S100A9, TNF, IL6, IL2, ATF3, SRF, PENK1, ADRB1, TACR1, CALB1, CALB2, CALR, IL6, PMAIP1, THBS1 and their equivalents or homologs.

It has been suggested that reduced cAMP induction has implications for the underlying causes of fragile X syndrome (FX) and autism spectrum disorders (see Kelley D J, Davidson R J, Elliott J L, Lahvis G P, Yin J C, Bhattacharyya A, The cyclic AMP cascade is altered in the fragile X nervous system, PLoS One. 2007 Sep. 26; 2(9):e931; Berry-Kravis E, Hicar M, Ciurlionis R, Reduced c relic AMP production in fragile X syndrome: cytogenetic and molecular correlations, Pediatr Res. 1995 November; 38(5):638-43) meaning that dysregulation of genes involved in cAMP pathway has strong impact on behavior. Our results indicate that SATB1 regulates the gene expression in the response to cAMP induction.

While diagnosis and detection of other neurological and psychiatric disorders may rely on gene amplification of certain genes, the present invention relies on the ectopic expression of SATB1 and/or SATB2 (sometimes collectively referred to herein as “SATB1/2”). In addition, any alternative forms of SATB1/2 present in the brain, such as a post-translationally modified version of SATB1/2, when expressed, is likely to result in an increase in neurological dysfunction and dysregulation of many genes involved in brain function. Alternatively, abnormal levels (e.g., elevated or decreased) of SATB1/2 also likely result in increases in neurological dysfunction and dysregulation of many genes involved in brain function. Because SATB1/2 is a gene regulator, mutated SATB1 or insufficient, modulated or reduced levels of SATB1/2 may be insufficient to turn on and organize the many genes involved in normal brain function in vivo.

In a preferred embodiment, because detection of SATB1/2 within bodily fluids such as blood cells by a blood test of a patient because taking a brain biopsy sample is likely infeasible. Therefore, detection of a variant form of SATB1/2 would be a useful marker for determining and diagnosing neurological dysfunction and psychiatric disorders. In one embodiment, to biochemically characterize variants of SATB1/2, we can use BUR affinity chromatography to purify SATB1/2 from a patient specimen using the established method as described in Kohwi-Shigematsu et al., Methods in Cell Biology 53: 324-352, 1998.

Specific region of modification in a variant SATB1/2 protein can be identified by techniques known and useful in the art, such as nuclear magnetic resonance (NMR), MALDI analysis (e.g., MALDI-TOF). This is similar to a case for a cancer-specific protein, PCNA observed by and as described or adapted from Bechtel P E, et al. who found “A unique form of proliferating cell nuclear antigen is present in malignant breast cells.” Cancer Res. 1998 Aug. 1; 58(15):3264-9. Methods can be also be used or adapted as described by Naryzhny SNA and Lee H, “Observation of multiple isoforms and specific proteolysis patterns of proliferating cell nuclear antigen in the context of cell cycle compartments and sample preparations,” Proteomics. 2003 June; 3(6):930-6, which describes data consistent with the idea that the existence of the different isoforms and specific proteolysis of PCNA are relevant to its functions in vivo. Both references are hereby incorporated by reference in their entirety.

Therefore reagents and tools can be created by methods known in the art based upon and to detect variants of SATB1 and/or SATB2 protein specifically expressed in brain cells for use in diagnosis and prognosis. In another embodiment, therapeutics can be made to provide or deliver normal SATB1/2 protein to promote its expression and increase its function to prevent or overcome neurological dysfunction and psychiatric disorders. In one embodiment, the normal SATB1/2 protein levels would be at normal levels after delivery or action by the therapeutic.

In one embodiment of the invention, methods for detection of SATB1/2 variant proteins is provided for use in diagnosis and prognosis of neurological disease, dysfunction and psychiatric disorders. In one embodiment, the neurological disease detected is Alzheimer's disease. In other embodiments, SATB1 is detected in other neurological diseases and psychiatric disorders including, but not limited to, mental retardation, epilepsy, attention-deficit disorder, depression, fragile X syndrome (FX) and autism spectrum disorders, and psychiatric disorders.

Note this is in contrast to what is observed in normal breast tissues. In normal breast tissues SATB1 expression is undetectable, and low to undetectable levels of SATB1 expression are detected in carcinoma originally diagnosed as moderately differentiated infiltrating ductal carcinomas and high levels of SATB1 expression can be detected in metastatic breast carcinomas. However, detection of SATB1 and/or SATB2 variants in tissues or bodily fluids may be diagnostic or prognostic of SATB1/2 related disease.

Thus, the invention encompasses likely SATB1 and/or SATB2 variants. In one embodiment, SATB1 and/or SATB2 variant is one which has at least about 80%, more preferably at least about 90%, and most preferably at least about 95% amino acid sequence identity to wild-type SATB1 and/or SATB2 amino acid sequence, and which contains at least one functional or structural characteristic of SATB1 and/or SATB2.

The invention also encompasses polynucleotides which encode SATB1 and/or SATB2. In a particular embodiment, the invention encompasses a polynucleotide sequence comprising the sequence of SEQ ID NO:3 which encodes SATB1, and SEQ ID NO:7 which encodes SATB2.

SATB1 is located on chromosome 3p23, GeneID 6304 and the Unigene Locus number is Hs.517717. Useful sequences for making probes and other sequences in the present invention include but are not limited, human SATB1 mRNA found at GenBank Accession No. NM_{—}002971.2 (GI:33356175) (SEQ ID NO:3), and human SATB1 protein sequence, GenBank Accession No. NP_{—}002962 (SEQ ID NO:4), all of which are hereby incorporated by reference.

SATB2 is located on chromosome 2q33, GeneID 23314 and the Unigene Locus number is Hs.517717. Useful sequences for making probes and other sequences in the present invention include but are not limited, human SATB2 mRNA found at GenBank Accession No. NM_{—}015265 XM_{—}031223 (GI:170016089) (SEQ ID NO:7), and human SATB2 protein sequence, GenBank Accession No. NP_{—}056080.1 (SEQ ID NO:8), all of which are hereby incorporated by reference.

In another embodiment, probes and primers to detect SATB1 and/or SATB2 and other sequences can be made using techniques well known in the art and the sequences provided including, Mouse Satb1 gene GenBank Accession Number: U05252 (SEQ ID NO:1) and Mouse Satb1 protein GenBank Accession Number: AAA17372.1 (SEQ ID NO:2) and Mouse SATB2 gene GenBank Accession Number: NM_{—}139146 (SEQ ID NO:5) and Mouse SATB2 protein GenBank Accession Number: NP_{—}056080.1 (SEQ ID NO:6).

The invention also encompasses a variant of a polynucleotide sequence encoding SATB1 and/or SATB2. In one particular embodiment, such a variant polynucleotide sequence will have at least about 80%, more preferably at least about 90%, and most preferably at least about 95% polynucleotide sequence identity to the polynucleotide sequence encoding SATB1. A particular aspect of the invention encompasses a variant of SEQ ID NO:3 which has at least about 80%, more preferably at least about 90%, and most preferably at least about 95% polynucleotide sequence identity to SEQ ID NO:3. In another particular embodiment, a variant polynucleotide sequence will have at least about 80%, more preferably at least about 90%, and most preferably at least about 95% polynucleotide sequence identity to the polynucleotide sequence encoding SATB2. A particular aspect of the invention encompasses a variant of SEQ ID NO:7 which has at least about 80%, more preferably at least about 90%, and most preferably at least about 95% polynucleotide sequence identity to SEQ ID NO:7. Any one of the polynucleotide variants described above can encode an amino acid sequence which contains at least one functional or structural characteristic of SATB1 and/or SATB2 proteins (SEQ ID NO:4 and SEQ ID NO:8, respectively).

It will be appreciated by those skilled in the art that as a result of the degeneracy of the genetic code, a multitude of polynucleotide sequences encoding SATB1 and/or SATB2, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring SATB1 and/or SATB2, and all such variations are to be considered as being specifically disclosed.

Although nucleotide sequences which encode SATB1 and/or SATB2 and its variants are preferably capable of hybridizing to the nucleotide sequence of the naturally occurring SATB1 and/or SATB2 under appropriately selected conditions of stringency, it may be advantageous to produce nucleotide sequences encoding SATB1 and/or SATB2 or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding SATB1 and/or SATB2 and its derivatives without altering the encoded amino acid sequences include the production of RNA transcripts having more desirable properties, such as a greater half-life, than transcripts produced from the naturally occurring sequence.

The invention also encompasses production of DNA sequences which encode SATB1 and/or SATB2 and SATB1 and/or SATB2 derivatives, variants or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding SATB1 and/or SATB2 or any fragment thereof. Amplification, sequencing and analysis of the SATB1 and/or SATB2 sequences, derivatives, variants or fragments may also be carried out.

Also encompassed by the invention are polynucleotide sequences that are capable of hybridizing to the claimed polynucleotide sequences and/or their variants, and, in particular, to those shown in SEQ ID NO:3 or 7, or a fragment of SEQ ID NO:3 or 7, under various conditions of stringency. (See, e.g., Wahl, G. M. and S. L. Berger (1987) Methods Enzymol. 152:399-407; Kimmel, A. R. (1987) Methods Enzymol. 152:507-511.)

Any method known in the art can be used to identify a single nucleotide polymorphism present in SATB1 and/or SATB2. In one embodiment, methods such as those described in Gresham et al., Science 31 Mar. 2006: Vol. 311. no. 5769, pp. 1932-1936, Science Express on 9 Mar. 2006, “Genome-Wide Detection of Polymorphisms at Nucleotide Resolution with a Single DNA Microarray,” using their SNPscanner algorithm can be used. In another embodiment, parallel genotyping as described by Hirschhorn et al. in PNAS Oct. 24, 2000 vol. 97 no. 22 12164-12169, which describes a method for parallel genotyping of SNPs, using single base extension-tag (SBE-TAGS) array on glass slides can be used. SNPs are genotyped by using bifunctional primers carrying a unique sequence tag in addition to a locus-specific sequence to identify a SNP.

In another embodiment, a screen can be made to identify clinically relevant SNPs in SATB1 and SATB2 that are involved in neurological function. A starting point is designing primers, probes and arrays for detecting SNPs in human SATB1 and SATB2 summarized in publicly available SNP databases, such as the NCBI SNP database, dbSNP, or the UCSC genome bioinformatics database (available online at the UCSC genome website). Currently in dbSNP, there are 481 SNPs found for human SATB1 and 992 SNPs found for human SATB2. Probes designed to detect each of the SNPs by microarray analysis or multiplex analysis on suspension array systems such as Luminex 100 can be made.

Further extensive sequencing can also be performed to find novel SNPs or feature polymorphisms in SATB1 and SATB2, which may be clinically relevant. Probes and primers found can be added to an array containing publicly available SNPs to provide a complete SNP array platform for screening patient samples.

After SNPs and genotypes to be detected are identified, detection will prove simple for one of ordinary skill in the art. Any number of techniques to detect the genotype of an individual by genotyping the individual at certain polymorphic sites can be used, including, but not limited to, the following.

The nucleotide can be determined by sequencing analysis after DNA samples are subjected to PCR amplification. Methods for DNA sequencing are well known and generally available in the art and may be used to practice some of the embodiments of the invention. The methods may employ such enzymes as the Klenow fragment of DNA polymerase I, Taq polymerase (Perkin Elmer), thermostable T7 polymerase (Amersham, Chicago, Ill.), phi29 enzyme (GE Healthcare) or combinations of polymerases and proofreading exonucleases. Preferably, the process is automated with machines such as thermocyclers, and sequencing machines and the amplified DNA is subjected to automated sequencing reactions, e.g., using a dye-primer cycle dideoxy terminator sequencing protocol. The sequencing reactions are then sequenced using any number of commercially available sequencing machines such as the ABI 377, 3700 or 3730 Sequence Analyzer (Applied Biosystems, Foster City, Calif.). In another embodiment, the PCR amplified DNA samples are processed and sequenced using the Roche 454 platform (454 Life Sciences, Branford Conn.) or Illumina sequencing technology (Illumina, Inc., San Diego, Calif.).

For example, in one embodiment, to identify clinically relevant SNPs (i.e. those SNPs that are associated with a mental disorder), blood samples from patients with mental disorders can be used, and a full sequence analysis performed for the cDNA encoding SATB1. Such sequencing analysis can be performed with several primer sets specific for amplifying different fragments of SATB1 cDNA. The amplified regions of SATB1 cDNA can be sequenced by a sequencer, such as the 3730 DNA Analyzer which is suited for medium-to-high throughput genetic analysis. With this sequencing analysis, DNA fragments generated from 96 blood samples could be simultaneously sequenced to detect SNPs.

In another embodiment, a suspension array systems such as the LUMINEX platform (Luminex Corp., Austin, Tex.) is used which relies on capture and hybridization assays using color-coated microspheres displaying ligands for high throughput detection and multiplex nucleic acid detection in the 96-well format. Oligonucleotides can be designed for multiplex analysis on LUMINEX 100. Primer extension assays can be performed involving Primer extension is a two step process that first involves the hybridization of a probe to the bases immediately upstream of the SNP nucleotide followed by a ‘mini-sequencing’ reaction, in which DNA polymerase extends the hybridized primer by adding a base that is complementary to the SNP nucleotide. This incorporated base is detected and determines the SNP allele (See Milani L, Syvänen A C, Genotyping single nucleotide polymorphisms by multiplex minisequencing using tag-arrays, Methods Mol. Biol. 2009; 529:215-29; and Syvänen A C, Accessing genetic variation: genotyping, single nucleotide polymorphisms, Nat Rev Genet. 2001 December; 2(12):930-42. Review). Design of oligonucleotides and reagents for such primer extension assays and services is also commercially available at PREMIER BioSoft International (Palo Alto, Calif.).

In another embodiment, techniques and methods of synthesizing and amplifying polynucleotides by ligation of multiple oligomers (LMO) onto a template-bound primer are also described by Akhavan-Tafti in U.S. Pat. Nos. 5,998,175; 6,001,614; 6,013,456; and 6,020,138, which are hereby incorporated by reference in their entirety. Short polynucleotides, 5 to 10 bases long, can be supplied as a library of oligonucleotides and are simultaneously ligated, using a suitable ligase enzyme, to a template-bound primer in a contiguous manner to produce a complementary strand of template polynucleotide. If the sequence to be synthesized is known, a set containing the minimum number of oligomers can be used and are then ligated by DNA Ligase in the correct order starting from the primer, uni- or bi-directionally, to produce the complementary strand of a single-stranded template sequence.

In another embodiment, sequence detection/amplification assays such as the INVADER assays which are commercially available from Third Wave Technologies (Madison, Wis.) to genotype samples. Such systems rely on an enzyme-substrate reaction to amplify signal generated when a perfect match with an (rare) allele of SATB1 and/or SATB2 is detected. See Dahlberg, J. et al., U.S. Pat. Nos. 5,846,717 and 5,888,780, which are hereby incorporated by reference in their entirety.

In another embodiment, methods that have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis) See U.S. Pat. Nos. 5,547,835; 6,221,601; 6,194,144 which are hereby incorporated by reference in their entirety. Other methods of SNP analysis are performed by companies such as Sequenom (San Diego, Calif.), which can genotype many samples very quickly and with great accuracy using non-sequencing methods such as MALDI-TOF, miniaturized chip-based array formats and mass spectrometry.

Other genotyping methods suited for detection of SNPs include, but are in no way limited to, LCR (ligase chain reaction), Gap LCR (GLCR), using allele-specific primers, mismatch detection assays, microsequencing assays, and hybridization assay methods. In one embodiment, high throughput array detection may be used, e.g., Affymetrix GeneChip® Targeted Genotyping Panels, Affymetrix, (Affymetrix, Inc., Santa Clara, Calif.).

In one embodiment, a PCR assay is used to detect SATB1 and/or SATB2 expression. Primers can be created using the nucleotide sequences of SATB1 (SEQ ID NO: 3) and SATB2 (SEQ ID NO: 7), or surrounding genomic sequence, to detect sequence amplification by signal amplification in gel electrophoresis. As is known in the art, primers or oligonucleotides are generally 15-40 bp in length, and usually flank unique sequence that can be amplified by methods such as polymerase chain reaction (PCR) or reverse transcriptase PCR. Primers to detect SATB1 and/or SATB2 expression can be created based upon genomic sequence containing and flanking SATB1 and/or SATB2.

For all PCR-based methods, primers may be designed using commercially available software, such as OLIGO 4.06 primer analysis software (National Biosciences Inc., Plymouth, Minn.) or another appropriate program, to be about 22 to 30 nucleotides in length, to have a GC content of about 50% or more, and to anneal to the template at temperatures of about 68° C. to 72° C.

The nucleotide sequences of the present invention can be engineered using methods generally known in the art in order to alter SATB1 and/or SATB2-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In another embodiment, nucleotide sequences encoding normal and variant SATB1 and/or SATB2 may be synthesized, in whole or in part, using chemical methods well known in the art. (See, e.g., Caruthers, M. H. et al. (1980) Nucl. Acids Res. Symp. Ser. 215-223, and Horn, T. et al. (1980) Nucl. Acids Res. Symp. Ser. 225-232.) Alternatively, SATB1 and/or SATB2 itself or a fragment thereof may be synthesized using chemical methods. For example, peptide synthesis can be performed using various solid-phase techniques. (See, e.g., Roberge, J. Y. et al. (1995) Science 269:202-204.) Automated synthesis may be achieved using a peptide synthesizer. Additionally, the amino acid sequence of SATB1 and/or SATB2, or any part thereof, may be altered during direct synthesis and/or combined with sequences from other proteins, or any part thereof, to produce a variant polypeptide.

In one embodiment of the invention, fragments of various lengths of SATB1 and/or SATB2 DNA may be placed onto solid supports for use in gene chips or other parallel formats for assay purposes. In general, these methods employ arrays of oligonucleotide probes that are complementary to targeted nucleic acid sequences, and allow for detection when the sample hybridizes to a probe on the array. In preferred embodiments, the nucleic acid sequences are SATB1 and/or SATB2 fragments of about 15-30 nucleotides in length, specifically sequences containing SATB1 and/or SATB2 variants or SNPs. See D. J. Lockhart, et al., “Expression monitoring by hybridization to high-density oligonucleotide arrays,” Nature Biotechnology, 14:1675-1680, December 1996, for useful methods and heuristics in designing oligonucleotide probes from SATB1 and/or SATB2 fragments.

Chips of various formats from companies such as Agilent Technologies (Palo Alto, Calif.) and Affymetrix (Santa Clara, Calif.) can be produced on a customized basis by various methods. Alternatively, DNA microarray chips are fairly inexpensive to make and assemble. Individual samples to be tested are then contacted with the oligonucleotide probes and the genotype of the sample can be determined based on detection of the hybridization between the probes and the sample. A suitable DNA micro-array is disclosed in Brown et al. U.S. Pat. No. 5,807,522.

Expression microarray analysis may be used to compare the global gene expression patterns between wild type and SATB1-null, or SATB1 variant expression as in Example 2. Such methods are now known in the art and are practiced more readily using microarray technology such as Affymetrix Gene Expression Analysis Arrays and Reagents (Affymetrix, Santa Clara, Calif.) and techniques such as comparative genomic hybridization (See Daniel Pinkel, et al., Array comparative genomic hybridization and its applications in cancer, Nature Genetics 37, S11-S17 (2005)).

Peptides comprising whole protein or fragments of SATB1, SATB2, and/or their variants, may be substantially purified by preparative high performance liquid chromatography. (See, e.g, Chiez, R. M. and F. Z. Regnier (1990) Methods Enzymol. 182:392-421.) The composition of the synthetic peptides may be confirmed by amino acid analysis or by sequencing. (See, e.g., Creighton, T. (1984) Proteins, Structures and Molecular Properties, WH Freeman and Co., New York, N.Y.).

The present embodiment encompasses a recombinant vector comprising a polynucleotide that is substantially homologous to any of the polynucleotides described herein, including regulatory sequences, coding sequences and polynucleotide constructs, as well as any SATB1 and/or SATB2 primer or probe. In a first preferred embodiment, a recombinant vector comprises expression vectors comprising either a regulatory polynucleotide of SATB1 and/or SATB2 or a coding nucleic acid of the present embodiment, or both. Within some embodiments, the expression vectors are employed in the in vivo expression of SATB1 and/or SATB2 in non-human animals. In other embodiments, the expression vectors are used for constructing transgenic animals and gene therapy.

Depending on the host organism or cell wherein the SATB1 and/or SATB2 gene will be expressed, one skilled in the art can adapt the recombinant vector to further comprise genetic elements, including but not limited to, an origin of replication in the desired host, suitable promoters and enhancers, any necessary ribosome binding sites, polyadenylation signal, splice donor and acceptor sites, transcriptional termination sequences, selectable markers and non-transcribed flanking sequences. Various types of gene delivery vectors can be used including, but definitely not limited to, plasmids, YACs (Yeast Artificial Chromosomes), BACs (Bacterial Artificial Chromosomes), bacterial vectors, bacteriophage vectors, viral vectors (for example, retroviruses, adenoviruses and viruses commonly used for gene therepy), non-viral synthetic vectors, and recombinant vectors, etc.

In bacterial systems, a number of cloning and expression vectors may be selected depending upon the use intended for polynucleotide sequences encoding SATB1 and/or SATB2. For example, routine cloning, subcloning, and propagation of polynucleotide sequences encoding SATB1 and/or SATB2 can be achieved using a multifunctional E. coli vector such as BLUESCRIPT (Stratagene) or PSPORT1 plasmid (GIBCO BRL). Ligation of sequences encoding SATB1 and/or SATB2 into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of SATB1 and/or SATB2 are needed, e.g. for the production of antibodies, vectors which direct high level expression of SATB1 and/or SATB2 may be used. For example, vectors containing the strong, inducible T5 or T7 bacteriophage promoter may be used.

Plant systems may also be used for expression of SATB1 and/or SATB2. Transcription of sequences encoding SATB1 and/or SATB2 may be driven viral promoters, e.g., the 35S and 19S promoters of CaMV used alone or in combination with the omega leader sequence from TMV. (Takamatsu, N. (1987) EMBO J. 6:307-311.) Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., Hobbs, S. or Murry, L. E. in McGraw Hill Yearbook of Science and Technology (1992) McGraw Hill, New York, N.Y.; pp. 191-196.)

In mammalian cells, a number of viral-based expression systems may be utilized. In cases where an adenovirus is used as an expression vector, sequences encoding SATB1 and/or SATB2 may be ligated into an adenovirus transcription/translation complex consisting of the late promoter and tripartite leader sequence. Insertion in a non-essential E1 or E3 region of the viral genome may be used to obtain infective virus which expresses SATB1 and/or SATB2 in host cells. (See, e.g., Logan, J. and T. Shenk (1984) Proc. Natl. Acad. Sci. 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, lentiviral-based expression and delivery systems or vesicles) for therapeutic purposes.

Suitable neurotrophic viral vectors include, but are not limited to adeno-associated viral vectors (AAV), herpes simplex viral vectors (U.S. Pat. No. 5,672,344) and lentiviral vectors. For example, a HIV-based lentiviral vector has recently been developed which, like other retroviruses, can insert a transgene into the nucleus of host cells (enhancing the stability of expression) but, unlike other retroviruses, can make the insertion into the nucleus of non-dividing cells. This lentiviral vector has been shown to stably transfect brain cells after direct injection, and stably express a foreign trans gene without detectable pathogenesis from viral proteins (see, Naldini, et al., Science, 272:263-267 (1996), the disclosure of which is incorporated by reference). Following the teachings of the researchers who first constructed the HIV-1 retroviral vector, those of ordinary skill in the art will be able to construct lentiviral vectors suitable for use in the methods of the invention (for more general reference concerning retrovirus construction, see, e.g., Kriegler, Gene Transfer and Expression, A Laboratory Manual, W. Freeman Co. (NY 1990) and Murray, E J, ed., Methods in Molecular Biology, Vol. 7, Humana Press (NJ 1991)).

Adenoviruses and AAV have been shown to be quite safe for in vivo use and have been shown to result in long-term gene expression in vivo; they are therefore preferred choices for use in the methods of the invention, where safety and long-term expression of nervous system growth encoding transgenes (persisting for longer than necessary to stimulate regrowth of injured or diseased neurons) is necessary. Those of ordinary skill in the art are familiar with the techniques used to construct adenoviral and AAV vectors and can readily employ them to produce vector compositions useful in the claimed invention (for reference, see, e.g., Straus, The Adenovirus, Plenum Press (NY 1934), pp. 451-496; Rosenfeld, et al., Science, 252:431-434 (1991); U.S. Pat. No. 5,707,618 [adenovirus vectors for use in gene therapy]; and U.S. Pat. No. 5,637,456 [method for determining the amount of functionally active adenovirus in a vector stock], the contents of each of which is incorporated herein to illustrate the level of skill in the art). In one embodiment, AAV of any serotype can be used. The serotype of the viral vector used in certain embodiments of the invention is selected from the group consisting from AAV1, AAV2, AAV3, AAV4, AAV5, AAV6, AAV7, and AAV8 (see, e.g., Gao et al. (2002) PNAS, 99:11854-11859; and Viral Vectors for Gene Therapy: Methods and Protocols, ed. Machida, Humana Press, 2003). Other serotype besides those listed herein can be used. Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome (Auricchio et al., (2001) Hum. Mol. Genet., 10(26):3075-81),

Lentiviral-based vectors such as HIV and FIV are currently at earlier stages of development but also are attractive candidates for in vivo gene therapy based upon stability of expression in vivo and safety profiles

In one embodiment, the vector comprises a transgene operably linked to a promoter. The transgene encodes a biologically active molecule, expression of which in the CNS results in at least partial correction for SATB1 and/or SATB2 expression, expression levels, or SATB1/2 related diseases.

A second embodiment comprises a host cell that has been transformed or transfected with one of the SATB1 and/or SATB2 polynucleotides described herein, in particular a polynucleotide comprising SEQ ID NO: 1, 3, 5 or 7 or a fragment or variant thereof. Appropriate host cells can be prokaryotic host cells, such as E. coli, Bacillus subtilis, Salmonella typhimurium, and strains from species including but not limited to, Pseudomonas, Streptomyces and Staphylococcus. Alternatively eukaryotic host cells can be used, including but not limited to, HeLa cells, HepG2 and other mammalian host cells. A preferred embodiment is a mammalian host cell comprising the SATB1 and/or SATB2 genomic region, wherein the SATB1 or SATB2 gene is disrupted by homologous recombination with a knockout vector.

In order to study the physiological and phenotypic consequences of a lack of synthesis of the SATB1 and/or SATB2 protein, both at the cellular level and at the organism level, the preferred embodiment also encompasses DNA constructs and recombinant vectors enabling conditional expression of a specific allele or genotype of the SATB1 and/or SATB2 genomic sequence such as a polynucleotide SEQ ID NO: 3 or 7 containing at least one single nucleotide polymorphism (SNP) or an SATB1 and/or SATB2 cDNA (SEQ ID NO: 1, 3, 5, 7) in a transgenic non-human animal. The embodiment also encompasses DNA constructs to generate animals having multiple copies of the SATB1 and/or SATB2 protein expressed and animals having no SATB1 and/or SATB2 protein that is expressed (“knock-out animals”).

The targeting construct can be built by various methods known in the art including but not limited to, PCR primers for integration by homologous recombination, using a repressor/marker promoter construct, Cre-LoxP system, and antisense constructs. One method preferred is using PCR products and primers to build the targeting construct. To build such a construct to make knockout non-human animals and cells, one would need the homology “arms” that flank each side of the sequence to be deleted or disrupted, and a selectable marker inserted between the arms to select for the marker function. The sequence to be deleted can be the whole SATB1 and/or SATB2 gene as the inventors did in Example 5, single or multiple exons, intervening genomic sequences, short peptide sequences and even single base pair deletions. After delivery of the construct into embryonic stem cells, selection for the marker permits gene deletion. Or for instance, SATB1 and/or SATB2 gene function can be disrupted by insertion of the selectable marker, by inserting insertion of the marker in the promoter, splice sites, or the open reading frame.

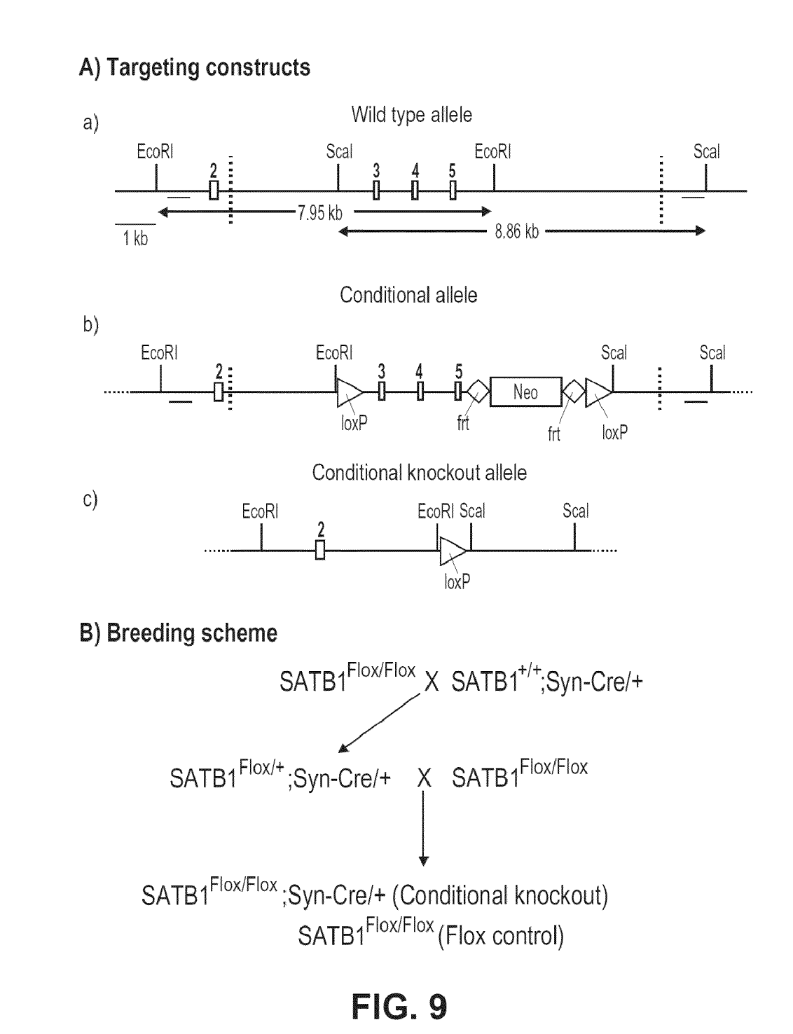

Thus in a specific embodiment, SEQ ID NO: 5, which is the isolated polynucleotide of the human SATB1 region, can be used to create constructs that includes SATB1 flanking sequence but does not include neighboring genes. In a preferred embodiment, the targeting construct to delete mouse SATB1 and/or SATB2 can be built using PCR products and primers made from SEQ ID NO: 5 and using the methods described in Example 5 and the constructs shown in FIG. 9.

In order to effect expression of the polynucleotides and polynucleotide constructs of the preferred embodiment, these constructs must be delivered to the host cell, where once it has been delivered to the cell, it may be stably integrated into the genome of the host cell and effectuate cellular expression. This delivery can be accomplished in vitro, for laboratory procedures for transforming cell lines, or in vivo or ex vivo, for the creation of therapies or treatments of diseases. Mechanisms of delivery include, but are not limited to, viral infection (where the expression construct is encapsulated in an infection viral particle), other non-viral methods known in the art such as, calcium phosphate precipitation, DEAE-dextran, electroporation, direct micro-injection, DNA-loaded liposomes, and receptor-mediated transfection of the expression construct. In a preferred embodiment, the delivery of the construct is by micro-injection into the appropriate host cell or by intravenous injection in the organism.

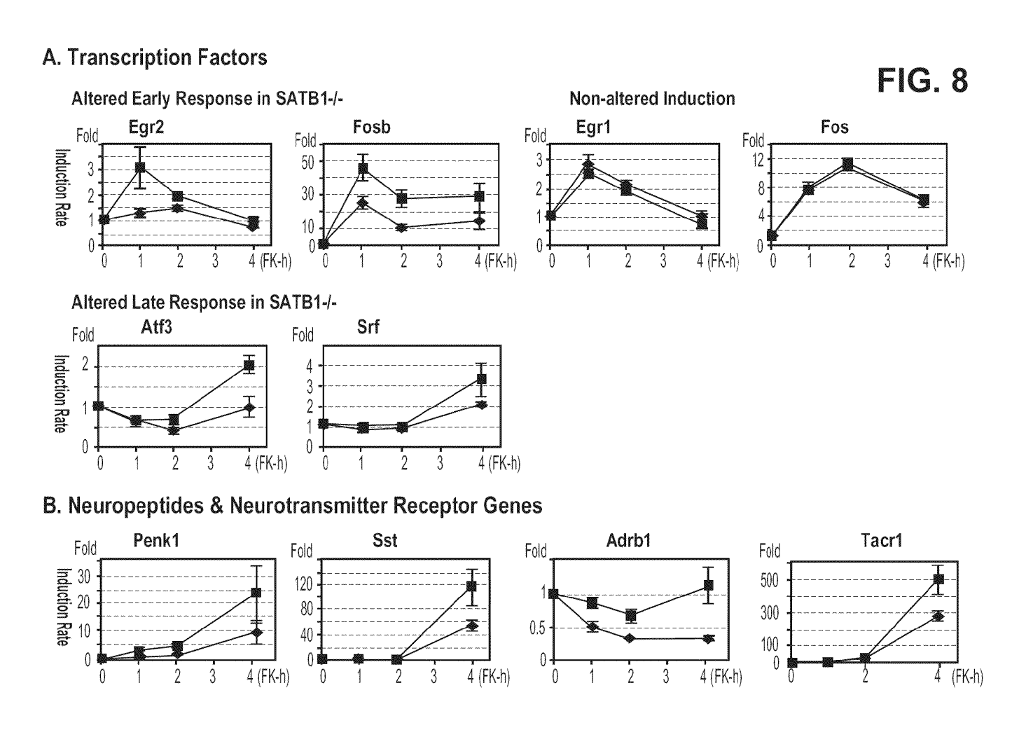

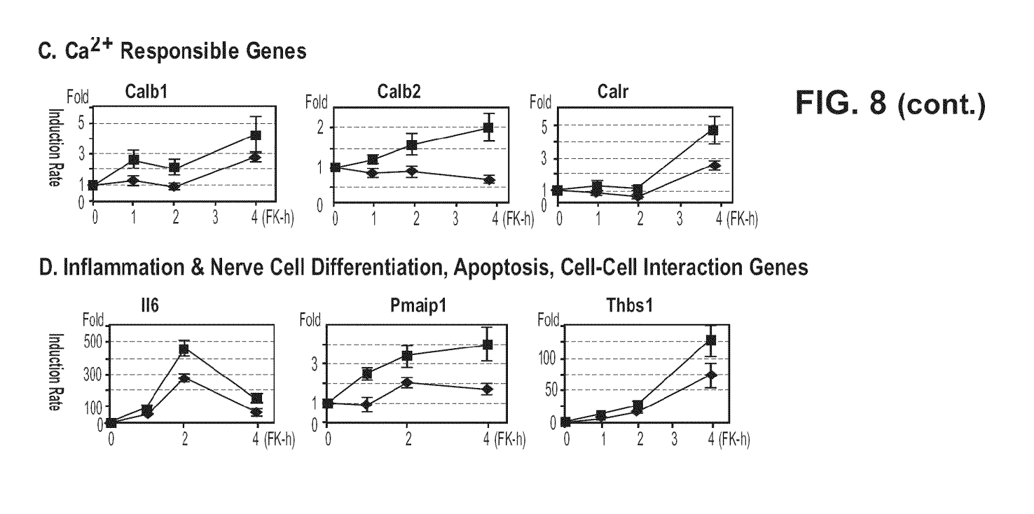

Thus, the preferred embodiment also provides non-human animals to assess SATB1 and/or SATB2 function. These non-human animals are preferably mammalian, even more preferably from the group consisting of mouse, rat, dog, chimpanzee, orangutan, baboon and macaque. These non-human animals are most preferably of the species Mus musculus, conditionally-expressing human SATB1 and/or SATB2 in cells expressing synapsin, as well as mice lacking Satb1 and/or SATB2 through standard mouse transgenic and gene knockout technologies (FIG. 8). Satb1 and/or SATB2 conditional knock-out animals and transgenic animals exhibit aggressive behavior and abnormal neurological function as described in Example 6.

An alternate embodiment also provides homozygous knockouts that are lacking Satb1 and/or SATB2 protein or lacking functional Satb1 and/or SATB2 protein. Transformed or transgenic cells, cell lines or non-human animals are obtained by homologous recombination of at least one Satb1 and/or SATB2 exon in embryonic stem cells, transfer of these stem cells to embryos, selection of the chimeras affected at the level of the reproductive lines, and growth of the said chimeras. Following successful germ-line transmission, heterozygous animals are then intercrossed.

To generate non-human animals which conditionally express SATB1 and/or SATB2, SEQ ID NO: 3 or 7, can be integrated into the genome of non-human embryos, thereby resulting in the knockout or expression of several copies of the human SATB1 and/or SATB2 gene by the non-human animals. In addition, transgenic animals such as rats and rabbits, or transgenic continuous cell lines can be made. Furthermore, transgenic animals can be made using cDNA encoding human SATB1 and/or SATB2, both in its wild type and variants as described herein.

Transgenic non-human animals over-expressing the SATB1 and/or SATB2 gene could be obtained by transfection of multiple copies of said SATB1 and/or SATB2 gene under the control of a strong promoter of an ubiquitous nature, or promoters selective for a type of tissue, preferably brain tissue.

In another embodiment, SATB1 conditional-null mice are created for use in a Cre-LoxP system. As shown in Example 5, these conditional KO mice were made by preparing ES cells containing a floxed Satb1 gene, i.e., Satb1 gene has its exons flanked by loxP sites (“flox” meaning flanked by loxP). We prepared mouse lines made from these ES cells, in which either one allele has the floxed Satb1 gene (heterozygous Satb1^{flox/+}) mice or both alleles have the floxed Satb1 gene (Satb1 conditional-null, or Satb1^{flox/flox}) mice after breeding, These mice have an almost completely wild-type allele. However, when Satb1^{flox/flox} mice of Satb1^{flox/+} mice are bred with transgenic mice expressing Cre recombinase driven by a cell-type specific promoter, some offspring will inherit both the floxed allele and the Cre-transgene. These mice will lack the Satb1 exons on one chromosome, due to the action of Cre on the remaining two loxP sites. Further crosses between Satb1^{flox/flox} mice and flox/Cre mice will result in a homozygous knockout for Satb1 gene in a cell-type specific manner. Such mice are useful in studying Satb1 function in any cell type of interest, using transgenic mice containing the corresponding cell type-specific promoter-driven Cre recombinase gene

By breeding mice, we have now established, mice in which Satb1 is deleted from most mature neurons of the post-natal brain where synapsin is expressed, using transgenic mice containing the Synapsin promoter driven-Cre recombinase gene. We have also established, where Satb1 is deleted from neurons in which CamKII is expressed. Such conditional-null mice, when tested in several memory tasks, such as object recognition, fear conditioning, and spatial navigation, allow the assessment of the role of Satb1 in learning and memory-associated behaviors.

This embodiment also provides non-human animals for further animal studies by pharmaceutical companies to study SATB1 and/or SATB2. Animal studies that explore the regulation and expression of SATB1 and/or SATB2, its interaction with other immediate early genes or other neurological proteins, production of antibodies for mutant and wild-type SatB1 and/or SATB2, and further in vivo study of Satb1 and/or SATB2. For example, mice lacking wild-type Satb1 and/or SATB2 may be exposed to various test substances to determine the neurological effect of the test substance on individuals having a non-wild-type Satb1 and/or SATB2 gene. If a certain drug is no longer able to work, it would indicate that Satb1 and/or SATB2 is needed for the given drug to exert its affect.

Preferably, said transformed cells or mammals of the preferred embodiment will be used as a model allowing, in particular, the selection of products which make it possible to combat the pathologies induced by loss of normal SATB1 and/or SATB2 function in normal post-natal brain function.

Immunological methods for detecting and measuring the expression of variant SATB1 and/or SATB2 using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on SATB1 and/or SATB2 is preferred, but a competitive binding assay may be employed. These and other assays are well known in the art. (See, e.g., Hampton, R. et al. (1990) Serological Methods, a Laboratory Manual, APS Press, St Paul, Minn., Section IV; Coligan, J. E. et al. (1997 and periodic supplements) Current Protocols in Immunology, Greene Pub. Associates and Wiley-Interscience, New York, N.Y.; and Maddox, D. E. et al. (1983) J. Exp. Med. 158:1211-1216).

Polyclonal and monoclonal antibodies can be made by well-known methods in the art. A preferred method of generating these antibodies is by first synthesizing SNP containing peptide fragments from variant SATB1 or SATB2 proteins. In another embodiment, these peptide fragments should cover unique regions in the SATB1 or SATB2 gene which are subject to altered post-translational modifications as compared to normal SATB1 which can contribute to altered SATB1/2 function in the brain. If a specific type of modification is found in SATB1 or SATB2 in the brain, a peptide with proper modification can be synthesized. Since synthesized peptides are not always immunogenic by their own, the peptides should be conjugated to a carrier protein before use. Appropriate carrier proteins include but are not limited to Keyhole limpet hemacyanin (KLH). The conjugated phospho peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal (e.g., anti-SATB1) antibodies can be purified by passing the harvested antibodies through an affinity column. Monoclonal antibodies are preferred over polyclonal antibodies and can be generated according to standard methods known in the art of creating an immortal cell line which expresses the antibody. In one embodiment, a SATB1 antibody as a control is an antibody of U.S. Pat. No. 5,869,621.

Nonhuman antibodies are highly immunogenic in human and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal anti-SATB1 antibodies.

In another embodiment, immunihistochemical analysis of brain tissue using an antibody against normal SATB1 or SATB2 will detect if there is normal SATB1 or SATB2 protein and/or function. However, immunohistochemical analysis of fixed neural tissue specimens and Western blot analysis of cell extracts using an antibody against variant SATBlor SATB2 will specifically detect the presence of neurological dysfunction in a given specimen.

Host cells transformed with nucleotide sequences encoding SATB1 and/or SATB2 may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode SATB1 and/or SATB2 may be designed to contain signal sequences which direct secretion of SATB1 and/or SATB2 through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (e.g., CHO, HeLa, MDCK, HEK293, and WI38), are available from the American Type Culture Collection (ATCC, Bethesda, Md.) and may be chosen to ensure the correct modification and processing of the foreign protein.

In another embodiment of the invention, natural, modified, or recombinant nucleic acid sequences encoding SATB1 and/or SATB2 may be ligated to a heterologous sequence resulting in translation of a fusion protein in any of the aforementioned host systems. For example, a chimeric SATB1 and/or SATB2 protein containing a heterologous moiety that can be recognized by a commercially available antibody may facilitate the screening of peptide libraries for inhibitors of SATB1 and/or SATB2 activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the SATB1 and/or SATB2 encoding sequence and the heterologous protein sequence, so that SATB1 and/or SATB2 may be cleaved away from the heterologous moiety following purification. Methods for fusion protein expression and purification are discussed in Ausubel, F. M. et al. (1995 and periodic supplements) Current Protocols in Molecular Biology, John Wiley & Sons, New York, N.Y., ch 10. A variety of commercially available kits may also be used to facilitate expression and purification of fusion proteins.

In a further embodiment of the invention, synthesis of radiolabeled variant SATB1 and/or SATB2 may be achieved in vitro using the TNT rabbit reticulocyte lysate or wheat germ extract systems (Promega, Madison, Wis.). These systems couple transcription and translation of protein-coding sequences operably associated with the T7, T3, or SP6 promoters. Translation takes place in the presence of a radiolabeled amino acid precursor, such as ³⁵S-methionine. For example, radiolabeled SATB1/2 (e.g., where the radiolabel is at the site of a SNP in either SATB1/2) may find use in model studies of neurological function or dysfunction. Such radiolabeled reagents also may find use in various imaging techniques, such as SPECT, CT and PET scans. As used herein and known in the art, SPECT is abbreviated for single photon emission tomography, CT for computed tomography and PET for positron emission tomography.

In another embodiment, the SNPs and genotypes of SATB1 and/or SATB2 will find use in any method known in the art to demonstrate a statistically significant correlation between a genotype and phenotype. The genetic analysis using the SNPs and genotypes that may be conducted include but are not limited to linkage analysis, population association studies, allele frequencies, genotype frequencies, and linkage disequilibrium.

Linkage analysis is based upon establishing a correlation between the transmission of genetic markers and that of a specific trait throughout generation within a family. Thus, the aim of linkage analysis is to detect marker loci that show co-segregation with a trait of interest. Linkage analysis correlating SATB1 and/or SATB2 SNPs and genotypes and the trait of neurological dysfunction to a psychiatric disorder, within families or people/ethnic groups are an aim of this invention.

Frequency of alleles and genotypes in a population is also another genetic analysis study contemplated by the invention. Using the genotyping methods described earlier, one skilled in the art can determine the frequency of SATB1 and/or SATB2 SNPs and genotypes in a given population. While several methods of estimating allele frequency are possible, genotyping individual samples is preferred over genotyping pooled samples due to higher sensitivity, reproducibility and accuracy. Furthermore, many genomic and large-scale sequencing centers enable rapid genotyping and haplotyping by sequencing methods and thereby provide rapid data production.

Association studies between SATB1 and/or SATB2 SNPs and any phenotype can also be performed on a random sample of people, anywhere from a few hundred to tens of thousands. After collecting various parameters for each individual participating the study, such as height, weight, psychiatric evaluation, medical history, etc., the sample group can be separated according to various genotypes at SATB1 and/or SATB2. Any repeated differences in the parameters in individuals that are observed are likely traits that are associated with one of the SATB1 and/or SATB2 genotypes. Examples show that there are differences in SATB1 and/or SATB2 regulated genes that are associated with neurological dysfunction, therefore, there are likely neurological disease correlations and associations that can be subject to study. Other parameters to observe include, but are not limited, presence of abnormal response to stimuli, anxiety levels, impaired neurological function, attention levels, depression, instances of neurological diseases, inflammatory response, immediate early gene expression levels, etc.

Statistical methods and computer programs useful for linkage analysis, genetic analysis and association studies are well-known to those skilled in the art. Any statistical tool useful to test for statistically significant associations between genotypes, haplotypes and phenotypes, comparisons and correlations between a biological marker and any physical trait, and frequency comparisons may be used. Statistical analyses can be carried out using the SAS computer program (SAS, Cary, N.C.) and similar programs. Comparison among different genotype groups can be made using Wilcoxon's test and the like. Allele frequencies should be compared using such tests as Fisher's exact test. To determine pairwise linkage disequilibrium (LD) between SNPs, genotype frequencies, estimations can be done using the Expectation-Maximization (EM) algorithm implemented in the computer program ARLEQUIN v. 2.0 ((Excoffier and Slatkin, Mol. Biol. Evol. 1995, 12 (5):921-927), and downloadable from http://lgb.unige.ch/arlequin/), an exploratory population genetics software environment.

Pair-wise measure of linkage disequilibrium (|D′|) can be calculated for all combinations of frequencies as described by R. C. Lewontin, Genetics 120, 849-52 (1988). A |D′| value of 1 indicates complete linkage disequilibrium between two markers.

Examples of useful statistical methods and techniques include Analysis of Variance (ANOVA), Fischer's test for pair-wise comparison and Wilcox's test, generally carried out using programs such as SPSS (Chicago, Ill.), STATVIEW and SAS (both available from SAS, Cary, N.C.).

Studies correlating the genotype with methods and treatments of neurological diseases and psychiatric diseases are also contemplated. Segregation of individuals in the study according to their response (e.g. increase in neurological function) to various drug therapies and combinations and then according to the SATB1 and/or SATB2 allele frequency. The result of stratification of population studies would enable doctors and medical care providers to prescribe therapy with greater accuracy, and with greater success rates. Thus, therapy prescribed would be appropriate for individuals based upon their genotypes and predicted response.

Upon genotyping or detection of a SNP or variant SATB1 and/or SATB2 in an individual and correlation with a specific disease or neutorlogical disorder, a course or therapeutic regimen can be adopted.

Therefore, in one embodiment, SATB1 and/or SATB2 or a fragment or derivative thereof may be administered to a subject to treat or prevent a neurodegenerative disorder. Such disorders can include, but are not limited to, akathesia, Alzheimer's disease, amnesia, amyotrophic lateral sclerosis, bipolar disorder, catatonia, cerebral neoplasms, dementia, depression, diabetic neuropathy, Down's syndrome, tardive dyskinesia, dystonias, epilepsy, Huntington's disease, peripheral neuropathy, multiple sclerosis, neurofibromatosis, Parkinson's disease, paranoid psychoses, postherpetic neuralgia, schizophrenia, and Tourette's disorder.

In another embodiment, a vector capable of expressing SATB1 and/or SATB2 or a fragment or derivative thereof may be administered to a subject to treat or prevent a neurodegenerative disorder including, but not limited to, those described above.

In a further embodiment, a pharmaceutical composition comprising a substantially purified SATB1 and/or SATB2 in conjunction with a suitable pharmaceutical carrier may be administered to a subject to treat or prevent a neurodegenerative disorder including, but not limited to, those provided above.

The invention further provides for compositions and methods to treat neural cells expressing variant SATB1. In one embodiment, the compound is a SATB1 and/or SATB2 variant inhibitor such as, an antisense oligonucleotide; a siRNA/shRNA olignonucleotide; a small molecule that interferes with SATB1 and/or function; a viral vector producing a nucleic acid sequence that inhibits SATB1 and/or SATB2; or an aptamer, wherein the SATB1 and/or SATB2 variant inhibitor specifically abrogates abnormal SATB1 and/or SATB2 function.

For example, such manipulation can be made using optimized shRNAs. Strong Pearson correlations between target expression levels and normalizing effects of shRNAs will indicate that expression levels determine the extent of response to target protein inhibitors. High throughput methods can be used to identify SATB1 inhibitors such as shRNA and/or small molecular inhibitor formulations to deliver SATB1 inhibitors efficiently to cultured neural cells. Variant-specific SATB1 inhibitory formulations will be preferentially effective against neurons that have variant-specific SATB1 expression and that these formulations will inhibit the. Effective formulations using such methods as described herein will be developed for clinical application.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of SATB1 and/or SATB2 may be produced using methods which are generally known in the art. In particular, purified SATB1 and/or SATB2 may be used to produce antibodies or to screen libraries of pharmaceutical agents to identify those which specifically bind SATB1 and/or SATB2. Antibodies to SATB1 and/or SATB2 may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are especially preferred for therapeutic use.

For the production of antibodies, various hosts including goats, rabbits, rats, mice, humans, and others may be immunized by injection with SATB1 and/or SATB2 or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic poilyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to SATB1 and/or SATB2 have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of SATB1 and/or SATB2 amino acids may be fused with those of another protein, such as KLH, and antibodies to the chimeric molecule may be produced.

Monoclonal antibodies to SATB1 and/or SATB2 may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; and Cole, S. P. et al. (1984) Mol. Cell. Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce SATB1 and/or SATB2-specific single chain antibodies. Antibodies with related specificity, but of distinct idiotypic composition, may be generated by chain shuffling from random combinatorial immunoglobulin libraries. (See, e.g., Burton D. R. (1991) Proc. Natl. Acad. Sci. 88:10134-10137.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between SATB1 and/or SATB2 and its specific antibody. A two-site, monoclonal-based immunoassay utilizing monoclonal antibodies reactive to two non-interfering SATB1 and/or SATB2 epitopes is preferred, but a competitive binding assay may also be employed. (Maddox, supra.)

In another embodiment of the invention, the polynucleotides encoding SATB1 and/or SATB2, or any fragment or complement thereof, may be used for therapeutic purposes. In one aspect, the complement of the polynucleotide encoding SATB1 and/or SATB2 may be used in situations in which it would be desirable to block the transcription of the mRNA. In particular, cells may be transformed with sequences complementary to polynucleotides encoding SATB1 and/or SATB2. Thus, complementary molecules or fragments may be used to modulate SATB1 and/or SATB2 activity, or to achieve regulation of gene function. Such technology is now well known in the art, and sense or antisense oligonucleotides or larger fragments can be designed from various locations along the coding or control regions of sequences encoding SATB1 and/or SATB2.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding SATB1 and/or SATB2. (See, e.g., Sambrook, supra; and Ausubel, supra.)

Genes encoding SATB1 and/or SATB2 can be turned off by transforming a cell or tissue with expression vectors which express high levels of a polynucleotide, or fragment thereof, encoding SATB1 and/or SATB2. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding SATB1 and/or SATB2. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing Co., Mt. Kisco, N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding SATB1 and/or SATB2.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding SATB1 and/or SATB2. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nature Biotechnology 15:462-466.)

Any of the therapeutic methods described above may be applied to any subject in need of such therapy, including, for example, mammals such as dogs, cats, cows, horses, rabbits, monkeys, and most preferably, humans.

In some embodiments, the SATB1 and/or SATB2 compositions are administered in combination with a second therapeutic agent for treating or preventing the diagnosed neurological or psychiatric disorder.

The SATB1 and/or SATB2 compositions, nucleic acids and the second therapeutic agent may be administered simultaneously or sequentially. For example, the inhibitory SATB1 nucleic acids may be administered first, followed by the second therapeutic agent. Alternatively, the second therapeutic agent may be administered first, followed by the inhibitory SATB1 nucleic acids. In some cases, the SATB1 and/or SATB2 compositions, nucleic acids and the second therapeutic agent are administered in the same formulation. In other cases the SATB1 and/or SATB2 compositions, nucleic acids and the second therapeutic agent are administered in different formulations. When the inhibitory SATB1 nucleic acids and the second therapeutic agent are administered in different formulations, their administration may be simultaneous or sequential.

In some cases, the SATB1 and/or SATB2 nucleic acids can be used to target therapeutic agents to cells and tissues expressing SATB1 and/or SATB2 that are related to immediate early gene expression and function. Thus it is contemplated that the present invention may be used in the diagnostic and therapeutic applications described herein for various psychiatric disorders and diseases related to neurological dysfunction.

An additional embodiment of the invention relates to the administration of a pharmaceutical or sterile composition, in conjunction with a pharmaceutically acceptable carrier, for any of the therapeutic effects discussed above. Such pharmaceutical compositions may consist of SATB1 and/or SATB2, antibodies to SATB1 and/or SATB2, and mimetics, agonists, antagonists, or inhibitors of SATB1 and/or SATB2. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

In certain embodiments, the nucleic acids encoding SATB1 and/or SATB2 peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses a wild-type or normal SATB1 and/or SATB2 peptides and nucleic acids of the present invention, thereby restoring normal expression of SATB1 and/or SATB2 in cells.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies.

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as described in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769). Additional suitable vectors include E1B gene-attenuated replicating adenoviruses described in, e.g., Kim et al., Cancer Gene Ther. 9(9):725-36 (2002) and nonreplicating adenovirus vectors described in e.g., Pascual et al., J. Immunol. 160(9):4465-72 (1998). Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding wild-type SATB1 nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

The SATB1 and/or SATB2 polynucleotides and compositions of the present invention, such as wild-type SATB1, also can be used to treat or prevent a variety of disorders associated with or caused by neurological dysfunction. The antibodies, peptides and nucleic acids are administered to a patient in an amount sufficient to elicit a therapeutic response in the patient (e.g., inhibiting the development, growth or metastasis of cancerous cells; reduction of tumor size and growth rate, prolonged survival rate, reduction in concurrent cancer therapeutics administered to patient). An amount adequate to accomplish this is defined as “therapeutically effective dose or amount.”

The antibodies, peptides and nucleic acids of the invention can be administered directly to a mammalian subject using any route known in the art, including e.g., by injection (e.g., intravenous, intrastriatal, intraperitoneal, subcutaneous, intramuscular, or intradermal), inhalation, transdermal application, rectal administration, or oral administration.

In other embodiments, such antibodies that specifically bind or inhibit variant SATB1 and/or SATB2, may be used therapeutically. Such use of antibodies has been demonstrated by others and may be useful in the present invention to inhibit or downregulate variant SATB1 and/or SATB2.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the antibodies, peptides and nucleic acids of the invention can be in any convenient manner, e.g., by injection, intrastriatal injection, intravenous and arterial stents (including eluting stents), catheter, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17th ed., 1989).

The present SATB1 and/or SATB2 compositions may be administered singly or in combination, and may further be administered in combination with other drugs known and determined by those familiar with the art to prevent or treat neurological disorders and disease. They may be conventionally prepared with excipients and stabilizers in sterilized, lyophilized powdered form for injection, or prepared with stabilizers and peptidase inhibitors of oral and gastrointestinal metabolism for oral administration.

The pharmaceutical forms suitable for injectable use include sterile aqueous solutions or dispersions and sterile powders for the extemporaneous preparation of sterile injectable solutions or dispersions (U.S. Pat. No. 5,466,468). The carrier can be a solvent or dispersion medium containing, for example, water, ethanol, polyol (e.g., glycerol, propylene glycol, and liquid polyethylene glycol, and the like), suitable mixtures thereof, and/or vegetable oils.

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease, and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, antibodies, polypeptides and nucleic acids of the present invention can be administered at a rate determined by the LD-50 of the polypeptide or nucleic acid, and the side-effects of the antibody, polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the SATB1 and/or SATB2 antibodies, peptides and nucleic acids of the present invention parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

In another embodiment, 5-20 micrograms of the present siRNA or antisense oligonucleotides can be suspended in 100 microliters of buffer such as PBS (phosphate buffered saline) for injecting into a subject intravenously to induce apoptosis of cancer cells. (See Slaton, Unger, Sloper, Davis, Ahmed, Induction of apoptosis by antisense CK2 in human prostate cancer xenograft model, Mol Cancer Res. 2004 December; 2(12):712-21.)

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580).

For intrastriatal administration, delivery of the therapeutic to a patient is carried out by sterotaxic instriatal injection or using a method of delivery such as convection enhanced delivery using a manual or non-manual pump for delivery, with or without the aid of a cannula. By convection enhanced delivery (CED) is meant infusion at a rate greater than 0.5.mu.l/min. Methods and compositions useful for application in the present invention are found in U.S. Pat. Nos. 7,182,944, 6,953,575, and US Patent Application Publication No. 20070259031, which describes compositions and methods for convection enhanced delivery of high molecular weight neurotherapeutics, all of which are hereby incorporated by reference in its entirety. For further teaching on the method of CED, see for example Saito et al., Exp. Neurol., 196:381-389, 2005; Krauze et al., Exp. Neurol., 196:104-111, 2005; Krauze et al., Brain Res. Brain Res. Protocol., 16:20-26, 2005; U.S. Patent Application Publication No. 2006/0073101; and U.S. Pat. No. 5,720,720, each of which is incorporated herein by reference in its entirety. See also Noble et al., Cancer Res. Mar. 1, 2006; 66(5):2801-6; Saito et al., J Neurosci Methods. Jun. 30, 2006; 154(1-2):225-32; Hadaczek et al., Hum Gene Ther. March 2006; 17(3):291-302; and Hadaczek et al., Mol. Ther. July 2006; 14(1):69-78, each of which is incorporated herein by reference in its entirety.

In another embodiment, multimodal probes comprising targeting, imaging, and therapeutic components are also contemplated for use in the invention. See WO 2009/045579 for description of probes which can be adapted for use in the present application. In some embodiments, probes and therapies should be able to cross the blood brain barrier.

Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most siRNA studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or stable siRNAs are readily soluble in aqueous solution and can be resuspended at concentrations as high as 2.0 mM. However, viscosity of the resultant solutions can sometimes affect the handling of such concentrated solutions.

In certain embodiments, the use of liposomes, nanocapsules, microparticles, microspheres, lipid particles, vesicles, and the like, are contemplated for the administration of the SATB1 and/or SATB2 compositions of the present invention. In particular, the compositions of the present invention may be formulated for delivery either encapsulated in or operatively attached to a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587). Se also U.S. Patent Application Publication No. 20070259031, which describes the use of liposomal-coated gadolinium for the use of imaging and therapeutics. All patents and patent applications cited herein regarding liposomes are hereby incorporated by reference.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation. In one embodiment, the present compositions are delivered using a synthetic low-density lipoprotein (sLDL) particle as described by WO/2007/145659, hereby incorporated by reference.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be easily made, as described (Couvreur et al., 1980; 1988; zur Muhlen et al., 1998; Zambaux et al. 1998; Pinto-Alphandry et al., 1995 and U.S. Pat. No. 5,145,684). Others have described nanoparticles in U.S. Pat. Nos. 6,602,932; 6,071,533.

It is further contemplated that the SATB1 inhibitors of the present invention is delivered to neuronal cells in a subject using other microparticles, nanostructures and nanodevices. For example, microspheres may be used such as those available from PolyMicrospheres, Inc. (Indianapolis, Ind.). For descriptions of drug delivery, see generally Alivisatos A P, Less is more in medicine, Understanding Nanotechnology, Warner Books, New York, 2002; Max Sherman, The World of Nanotechnology, US Pharm. 2004; 12:HS-3-HS-4; Brannon-Peppas and Blanchette, Nanoparticle and targeted systems for cancer therapy, Advanced Drug Delivery Reviews, Intelligent Therapeutics: Biomimetic Systems and Nanotechnology in Drug Delivery, Volume 56, Issue 11, 22 September 2004, Pages 1649-1659; and D. M. Brown, ed., Drug Delivery Systems in Cancer Therapy, Humana Press, Inc., Totowa, N.J. 2004, including Chapter 6: Microparticle Drug Delivery Systems by Birnbaum and Brannon-Peppas, pp. 117-136, all of which are hereby incorporated by reference.

In another embodiment, nanoparticles for targeting, imaging and therapeutic delivery may be employed. For example, imaging of the brain in a subject to detect variant SATB1/2 or SATB1/2 dysfunction can be carried out using nanoparticles comprising a material useful for detection using imaging techniques such as magnetic resonance (MR), near infrared (NR), composite tomography, PET (positron emission tomography).

In another embodiment, antibodies which specifically bind SATB1 and/or SATB2 or their variants may be used for the diagnosis of disorders characterized by expression of SATB1 and/or SATB2, or in assays to monitor patients being treated with SATB1 and/or SATB2 or agonists, antagonists, or inhibitors of SATB1 and/or SATB2 and their variants. Antibodies useful for diagnostic purposes may be prepared in the same manner as described above for therapeutics. Diagnostic assays for SATB1 and/or SATB2 include methods which utilize the antibody and a label to detect SATB1 and/or SATB2 in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring SATB1 and/or SATB2, including ELISAs, RIAs, and FACS, are known in the art and provide a basis for diagnosing altered or abnormal levels of SATB1 and/or SATB2 expression. Normal or standard values for SATB1 and/or SATB2 expression are established by combining body fluids or cell extracts taken from normal mammalian subjects, preferably human, with antibody to SATB1 and/or SATB2 under conditions suitable for complex formation The amount of standard complex formation may be quantitated by various methods, preferably by photometric means. Quantities of SATB1 and/or SATB2 expressed in subject, control, and disease samples from biopsied tissues are compared with the standard values. Deviation between standard and subject values establishes the parameters for diagnosing disease.

In another embodiment of the invention, the polynucleotides encoding SATB1 and/or SATB2 may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of SATB1 and/or SATB2 may be correlated with disease. The diagnostic assay may be used to determine absence, presence, and excess expression of SATB1 and/or SATB2, and to monitor regulation of SATB1 and/or SATB2 levels during therapeutic intervention.

In one aspect, hybridization with PCR probes which are capable of detecting polynucleotide sequences, including genomic sequences, encoding SATB1 and/or SATB2 or closely related molecules may be used to identify nucleic acid sequences which encode SATB1 and/or SATB2. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding SATB1 and/or SATB2, allelic variants, or related sequences.

Probes may also be used for the detection of related sequences, and should preferably have at least 50% sequence identity to any of the SATB1 and/or SATB2 encoding sequences. The hybridization probes of the subject invention may be DNA or RNA and may be derived from the sequence of SEQ ID NO:1, SEQ ID NO:3, SEQ ID NO:5 or SEQ ID NO:7 or from genomic sequences including promoters, enhancers, and introns of the SATB1 and/or SATB2 gene.

Means for producing specific hybridization probes for DNAs encoding SATB1 and/or SATB2 include the cloning of polynucleotide sequences encoding SATB1 and/or SATB2 or SATB1 and/or SATB2 derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences containing variants of SATB1 and/or SATB2 (e.g., SNPs of SATB1/2) may be used for the diagnosis of a disorder associated with expression of SATB1 and/or SATB2. Examples of such a disorder include, but are not limited to, neurodegenerative disorders such as akathesia, Alzheimer's disease, amnesia, amyotrophic lateral sclerosis, attention-deficit disorder, bipolar disorder, catatonia, cerebral neoplasms, dementia, depression, diabetic neuropathy, Down's syndrome, tardive dyskinesia, dystonias, epilepsy, Huntington's disease, peripheral neuropathy, multiple sclerosis, neurofibromatosis, Parkinson's disease, paranoid psychoses, postherpetic neuralgia, schizophrenia, and Tourette's disorder; and cancers such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding SATB1 and/or SATB2 may be used in Southern or Northern analysis, dot blot, or other membrane-based technologies; in PCR technologies; in dipstick, pin, and ELISA assays; and in microarrays utilizing fluids or tissues from patients to detect altered SATB1 and/or SATB2 expression. Such qualitative or quantitative methods are well known in the art.

In a particular aspect, the nucleotide sequences encoding variant SATB1 and/or SATB2 may be useful in assays that detect the presence of associated disorders, particularly those mentioned above. The nucleotide sequences encoding variant SATB1 and/or SATB2 may be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding SATB1 and/or SATB2 in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of variant SATB1 and/or SATB2, a normal or standard profile for expression is established. This may be accomplished by combining body fluids or cell extracts taken from normal subjects, either animal or human, with a sequence, or a fragment thereof, encoding SATB1 and/or SATB2, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of a relatively high amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding SATB1 and/or SATB2 may involve the use of PCR. These oligomers may be chemically synthesized, generated enzymatically, or produced in vitro. Oligomers will preferably contain a fragment of a polynucleotide encoding SATB1 and/or SATB2, or a fragment of a polynucleotide complementary to the polynucleotide encoding SATB1 and/or SATB2, and will be employed under optimized conditions for identification of a specific gene or condition. Oligomers may also be employed under less stringent conditions for detection or quantitation of closely related DNA or RNA sequences.

Methods which may also be used to quantitate the expression of SATB1 and/or SATB2 include radiolabeling or biotinylating nucleotides, coamplification of a control nucleic acid, and interpolating results from standard curves. (See, e.g., Melby, P. C. et al. (1993) J. Immunol. Methods 159:235-244; and Duplaa, C. et al. (1993) Anal. Biochem. 212:229-236.) The speed of quantitation of multiple samples may be accelerated by running the assay in an ELISA format where the oligomer of interest is presented in various dilutions and a spectrophotometric or colorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.)

In another embodiment of the invention, nucleic acid sequences encoding variant SATB1 and/or SATB2 may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Heinz-Ulrich, et al. (1995) in Meyers, R. A. (ed.) Molecular Biology and Biotechnology, VCH Publishers New York, N.Y., pp. 965-968.) Examples of genetic map data can be found in various scientific journals or at the Online Mendelian Inheritance in Man (OMIM) site. Correlation between the location of the gene encoding SATB1 and/or SATB2 on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., SATB1 in Chromoseome 1, any sequences mapping to that area may represent associated or regulatory genes for further investigation. (See, e.g., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, SATB1 and/or SATB2, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between SATB1 and/or SATB2 and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate, such as plastic pins or some other surface. The test compounds are reacted with SATB1 and/or SATB2, or fragments thereof, and washed. Bound SATB1 and/or SATB2 is then detected by methods well known in the art. Purified SATB1 and/or SATB2 can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding SATB1 and/or SATB2 specifically compete with a test compound for binding SATB1 and/or SATB2. In this manner, antibodies can be used to detect the presence of any peptide which shares one or more antigenic determinants with SATB1 and/or SATB2.

In additional embodiments, the nucleotide sequences which encode SATB1 and/or SATB2 may be used in any molecular biology techniques that have yet to be developed, provided the new techniques rely on properties of nucleotide sequences that are currently known, including, but not limited to, such properties as the triplet genetic code and specific base pair interactions.

Diagnostic/prognostic kit: The present invention further provides kits for use within any of the above diagnostic/prognostic methods. Such kits typically comprise two or more components necessary for performing a diagnostic/prognostic assay. Components of the kit may be compounds, reagents, containers and/or equipment. For example, one container within a kit may contain an antibody against SATB1 (normal version) and an antibody against variant SATB1. In one embodiment, the kit contains buffers to dilute variant SATB1 or SATB2 antibodies, fluorescent dye-conjugated secondary antibodies (anti-mouse or anti-rabbit) to detect variant SATB1 or SATB2 signals. In another embodiment, the kit contains buffers, reagents, primers and nucleotides for SNP detection array for detection of SATB1 and/or SATB2 SNPs. One or more additional containers may enclose elements, such as reagents or buffers, to be used in the assay. Such kits may also, or alternatively, contain a detection reagent as described above that contains a reporter group suitable for direct or indirect detection of antibody binding. In one embodiment, the kit can contain primers, or solution primers to each known SNP in SATB1 and/or SATB2, and buffers and solutions for PCR or sequencing for genotype detection.

Kits for therapeutic uses. Thus, the subject compositions of the present invention may be provided, usually in a lyophilized form, in a container. The SATB1 and/or SATB2 variant antibodies, chemicals, and/or nucleic acids described herein are included in the kits with instructions for use, and optionally with buffers, stabilizers, biocides, and inert proteins. Generally, these optional materials will be present at less than about 5% by weight, based on the amount of polypeptide or nucleic acid, and will usually be present in a total amount of at least about 0.001% by weight, based on the polypeptide or nucleic acid concentration. It may be desirable to include an inert extender or excipient to dilute the active ingredients, where the excipient may be present in from about 1 to 99% weight of the total composition. The kits may further comprise a second therapeutic agent.

### EXAMPLES

### Example 1

### SATB1 and/or SATB2 Expression in Mature Neurons in Postnatal Brain Subregions

To study the role of SATB1 in postnatal mouse brain, we first determined the expression patterns of SATB1 as well as SATB2 in the coronal sections of P13 wild-type mouse brain, using immunostaining and mRNA in situ hybridization (mRNA FISH) methods.

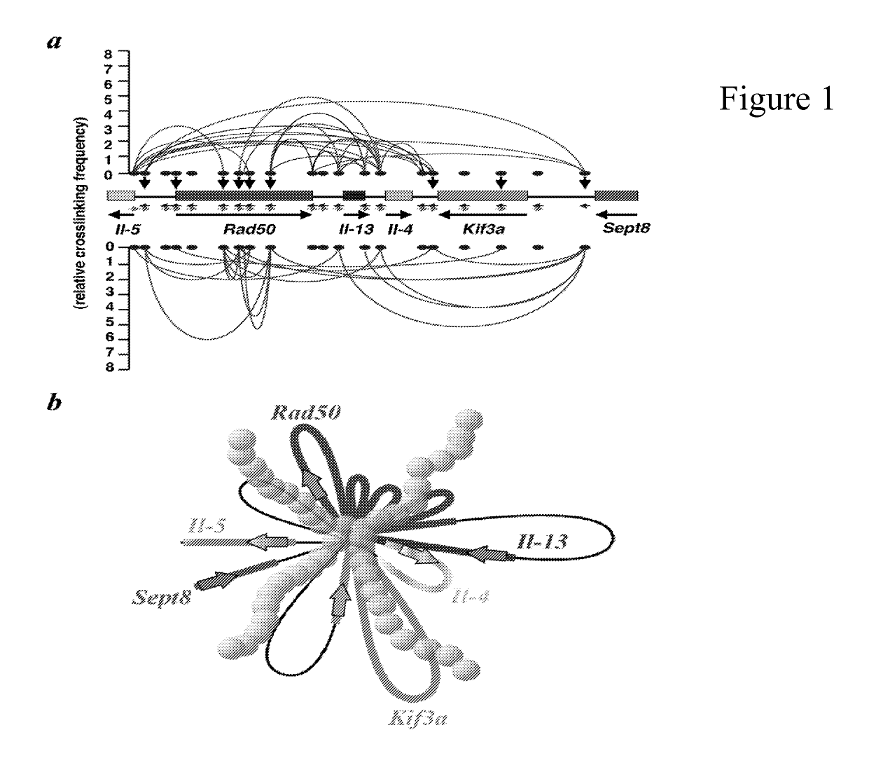

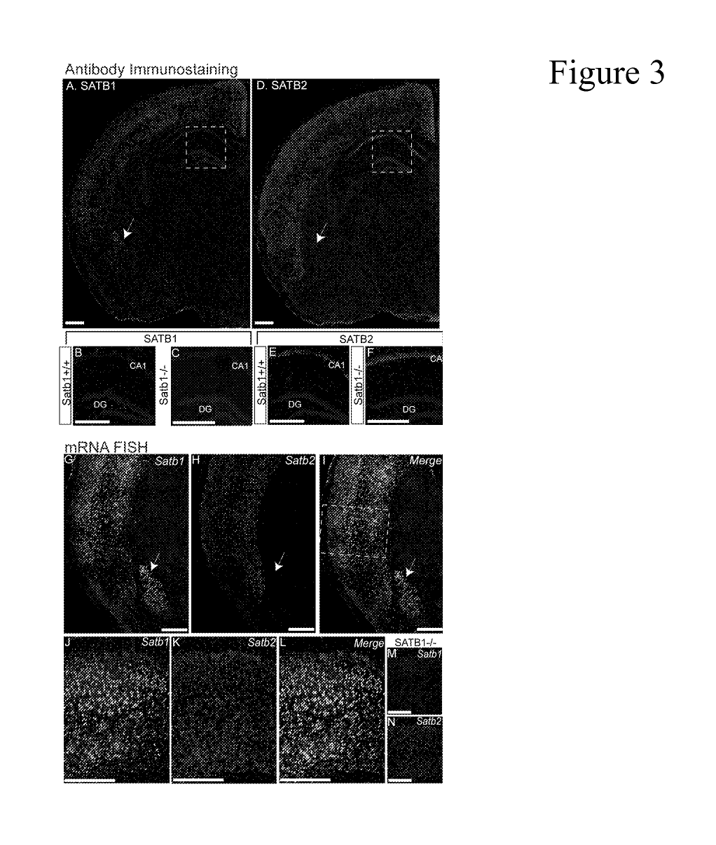

We employed antibody specific for SATB1 (FIG. 3A) or SATB2 (FIG. 3D). Sections of SATB1 knockout mouse brain were used to confirm the specificity of anti-SATB1 antibody (FIG. 3C). In wild type brain, we detected strong and widely distributed SATB1-specific signals in the cortex and amygdala (FIG. 3A). Additionally, some hilar cells in the dentate gyms (DG) and some cells in radial and stratum oriens layers of the hippocampus expressed SATB1 (FIGS. 3A, 3B). We also detected weak signals in some cells in hypothalamus (FIG. 3A). Within the amygdala, SATB1 was most abundantly expressed in the lateral and medial (indicated with arrow on FIG. 1A), but to a lesser extent in basomedial nuclei. SATB2, on the other hand, is found expressed at high levels in the cortex (FIG. 3D) including the outer and inner most layers where SATB1 expression is minimal. In contrast to SATB1, SATB2 is expressed in the CA1 region of hippocampus (FIG. 3E) but signal was absent in the hilar cells in the dentate gyms. SATB2 was totally absent in the amygdala (FIG. 3D). In SATB1-null brain the loss of SATB1 expression is not compensated by upregulation or induction of SATB2 in otherwise SATB1-specific areas like amygdala or dentate gyms (FIG. 3F and data not shown). The only region where both SATB1 and/or SATB2 expression was found was in the cortex.

To study whether any cells in the cortex express both SATB1 and/or SATB2, we employed mRNA FISH (FIG. 3G-3N). The specificity of Satb1 or SATB2 antisense probe was confirmed by using sense probe and/or sections form SATB1 knockout brain (FIG. 3M and data not shown). By using mRNA FISH we confirmed that cortex is the only region where Satb1 and/or SATB2 mRNA expression overlaps (FIG. 3G-3I). In the cortex, a there exists a subpopulation of cells that express both Satb1 and/or SATB2 mRNAs. However, a large number of cells express either Satb1 or SATB2, suggesting specific roles of for each member of SATB1 family in the cortex (FIG. 3J-3L).

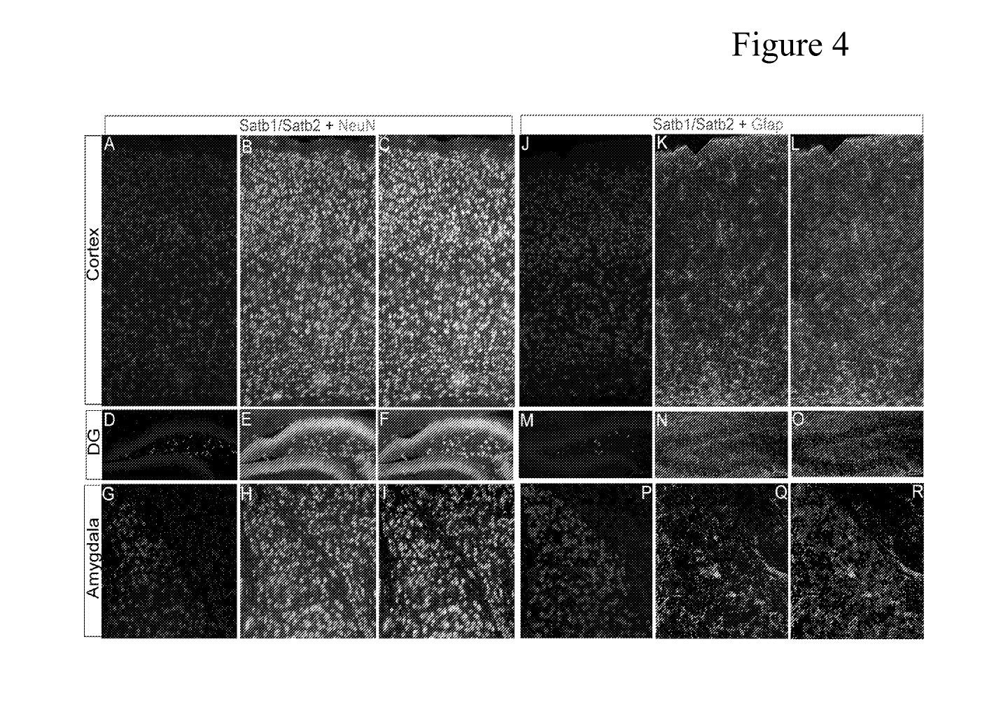

To determine which types of cells express SATB1 family proteins, double immunostaining with antibody recognizing both members of SATB family and antibody for neuronal marker NeuN (FIG. 4A-4I) or astrocyte marker Gfap (FIG. 4J-4R) was used. All cells expressing SATB family proteins were also NeuN-positive in all brain areas tested, including the cortex (FIG. 4A-4C), amygdala (FIG. 4D-4F) and dentate gyms (FIG. 4G-4I). No co-expression of Gfap with SATB family proteins was detected (FIG. 4J-4R). This data indicates that in the postnatal brain SATB1 and/or SATB2 are expressed only by postmitotic neurons.

Age-Dependent Expression of SATB1 and/or SATB2

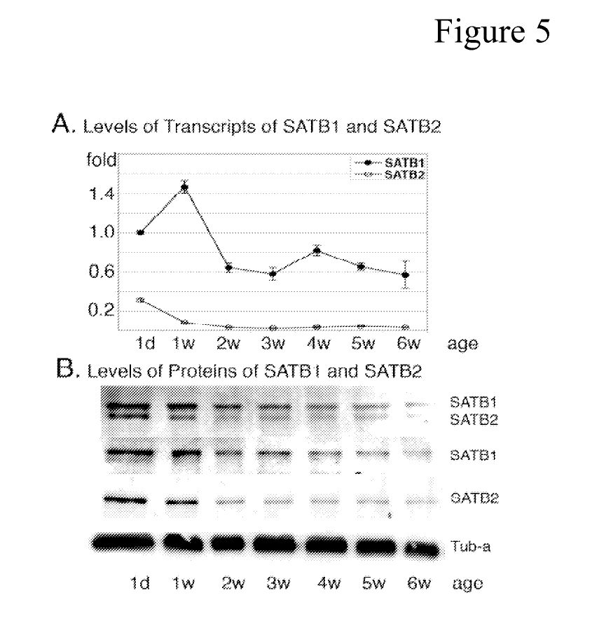

We examined mRNA and protein expression levels of SATB1 and/or SATB2 in postnatal cortex at different ages from day 1 to 6 weeks (FIG. 5). By a SuperArray designed to include SATB1/2 probes which give accurate relative quantification of SATB1 and/or SATB2 mRNA, we found that SATB1 mRNA is much greater than SATB2 mRNA at day 1. SATB1 mRNA reaches a peak at 1 week, followed by a reduction to 60-70% that of SATB1 mRNA level at day 1. Although SATB1 remains at that level through 6 weeks postnatally, SATB2 mRNA was greatly reduced after 1 week. The observed changes in the mRNA levels of SATB1/2 were confirmed at the protein levels by Western analysis using specific antibodies. The results show that SATB1 is the major SATB family protein expressed in postnatal cortex.

### Materials and Methods

Tissue processing. All mice were euthanized by CO₂ gas, brains for further RNA or protein extraction purpose were removed quickly and freezed down on dry ice. For brain section preparation, mice were perfused transcardinally with RNAase-free saline, brains were removed quickly and freezed down in isopentane on dry ice. Frozen tissue was cut into 10 μm thick coronal sections on cryostat. Mice were treated according to Lawrence Berkeley National Laboratory's Animal Care and Use Program, the animal protocol was approved by the Animal Welfare and Regulatory Committee at Lawrence Berkeley Laboratory (AWRC number 12501).

Immunofluorescence staining. 10 μm thick fresh frozen sections were fixed in 4% buffered paraformaldehyde, incubated with blocking solution 30 min (1% BSA, 0.1% cold fish gelatin, 0.1% Triton X-100, 0.05% Tween 20 in PBS), followed by incubation with primary antibody at 4° C. overnight and with proper secondary antibody at room temperature for 1 h. Following dilutions for primary antibodies were used: mouse anti-NeuN mAb (1:500, Chemicon), mouse anti-Gfap mAb (1:500, Chemicon), rabbit anti-Satb1 polyclonal Ab 1583 (1:2000, ref Dickinson), rabbit anti-SATB2 (1:1500).

### Example 2

### Identification of Genes Regulated by SATB1 in Postnatal Brain

Expression microarray analysis was used to compare the global gene expression patterns between wild type and SATB1-null mouse brains. Total RNA from 3 different P13 knockout and wild type littermate cortexes was extracted and individually subjected to Affymetrix array hybridization. A total of 109 genes were significantly altered by more than 1.4 fold between SATB1-null and wild type samples(p-value <0.05, 38 (35%) genes down-regulated and 71 (65%) up-regulated by SATB1). A representative gene list is shown (FIG. 6A, a full list of genes is not shown). Using the National Institutes of Health's DAVID software, the genes (p-value <0.05 and fold change ≧1.3) were classified into Gene Ontology (GO) categories. Interestingly, genes up-regulated by SATB1 are enriched in genes involved in transcription and in neuronal activity and development, while SATB1 downregulated genes are significantly represented by transport, cell organization and signaling (FIG. 6B). These results indicate that ablation of SATB1 affects a variety of different biological processes in postnatal mouse brain.

mRNA Fluorescent in situ Hybridization. To amplify SATB1 or SATB2 specific cDNA fragments, following primers were used:

(SEQ ID NO: 9)
SB1_5UTR_IS-F 5′-GGGAAGAGAAAATAATACA-3,
(SEQ ID NO: 10)
SB1_5UTR_IS-R 5′-TTTCCTAAGGTTGGTTTTC-3′,
(SEQ ID NO: 11)
SB2_5UTR_IS-F 5′-ATCATCATCATAACAACCATCTCC-3′,
(SEQ ID NO: 12)
SB2_5UTR_IS-R 5′-GCTCCAGCCGGGCCACCTTCAC-3′,
(SEQ ID NO: 13)
SB2_3UTR_IS-F 5′-GAGATGTACAAAGTGGAGGCTGAG-3′,
(SEQ ID NO: 14)
SB2_3UTR_IS-R 5′-CTGTGAAGTGGTATTAGTTTTTAT-3′

Linearized plasmids containing cloned cDNA fragments were used as templates for antisense and sense cRNA probe synthesis with T7 and T3 polymerase (Promega) and premixed RNA-labeling nucleotide mixes containing digoxigenin or fluorescein (Roche Molecular Biochemicals). The yield and integrity of cRNA riboprobes was confirmed by gel electrophoresis. 10 μm thick fresh frozen sections were fixed in 4% buffered paraformaldehyde and incubated twice with 1.35% triethanolamine (pH 8.0) and 0.25% acetic anhydride for 5 min. Sections were then incubated with prehybridization buffer containing 50% formamide, 5×SSC and salmon sperm DNA 40 μg/ml for 3-5 hours at room temperature. We diluted fluorescein-labeled Satb1 5′UTR-specific and dioxygenin-labeled SATB2 3′UTR and 5′UTR specific riboprobes in hybridization buffer (50% formamide, 1×Denhardt's solution, 3×SSC, 10 mM EDTA, 10% Dextran, 0.5 mg/ml yeast tRNA, 0.5 mg/ml salmon sperm DNA), heated the mixture for 6 min at 80° C. and added it to sections. We did hybridization at 65° C. for 16 hours. We washed hybridized sections twice with solution containing 50% formamide and 1×SSC for 20 min at 65° C., followed by washes with 0.2×SSC for 30 min at 65° C. and for 10 min at room temperature. Subsequently, sections were eliquilibrated in TN solution (100 mM Tris-HCl (pH 8.0), 150 mM NaCl) and blocked with TN solution containing 0.5% blocking reagent (Perkin Elmer Life Science) for 30 min. We first visualized the Satb1-specific signal. We incubated slides with rabbit antibody against fluorescein conjugated to poroxydase (1:500, Roche) at room temperature for 1 h, washed slides with TN buffer containing 0.05% Tween20 and visualized the signal by using Fluorescein Tyramide (TSA-Plus Fluorescence System, Perkin Elmer Life Science). We quenched the remaining peroxydase activity by incubating slides with 3% H₂O₂ solution in PBS for 30 min. To generate SATB2-specific signal, we used rabbit antibody against dioxygenin conjugated to peroxidase (1:500, Roche) and Cy5 Tyramide (TSA-Plus Fluorescence System, Perkin Elmer Life Science) as described above. Slides were mounted in fluorescent mounting medium containing DAPI (Vector Laboratories).

Microarry Analysis. We prepared total RNA from whole cortex of three 13-days-old Satb1-knockout and three wild-type littermate mice using Tri Reagent (Sigma) and further purified it with RNeasy mini kit (Qiagen). The concentration and purity of total RNA was measured by spectrophotometry at OD 260/280 and the integrity was assessed using 1.2% agarose gel. We used one microgram of total RNA to generate biotin-labeled cRNA with GeneChip One-Cycle Target Labeling system from Affymetrix, in accordance with manufacturer's protocol. We used 10 μg of biotin-labeled cRNA for fragmentation and sequential hybridization onto Affymetrix GeneChip Mouse Genome 430 2.0 arrays (Affymetrix). We used GeneSpring GX 7 software (Silicon Genetics, Redwood City, Calif.) for normalization of raw data and further analysis. To compare individual expression values across arrays, we normalized raw intensity data from each gene to the median intensity of the array. Only genes that had values greater than background intensity in at least 3 out of 6 arrays were used for further analysis. We used parametric test and all available error estimates with p-value cut off of 0.05 to generate list of genes significantly changed between wild-type and knockout samples. We additionally filtered obtained gene list based on fold change. All genes with fold change ≧1.3 were submitted for functional annotation based on the Gene Ontology (GO) classification performed with National Institutes of Health's DAVID tool (Dennis 2003).

RNA Isolation and Quantitative Real-Time PCR. We extracted total RNA from whole cortex of P13 Satb1 knockout and wild-type mice and evaluated it's quality as described above. We used 2 μg of each of total RNA for first strand cDNA synthesis with oligo(dT)₁₅ primer and Superscript II RNaseH-reverse transcriptase (Invitrogen), by following manufacturer's protocol. Primers for qRT-PCR were designed using Primer Express 3.0 software (Applied Biosystems) and sequences are available upon request. qRT PCRs were performed using Power SYBR Green protocol (Applied Biosystems) and ABI 7500 Fast Real-time PCR System. All reactions were done in three repeats with the following cycling protocol: 50° C. for 2 min, 95° C. for 10 min, 40 cycles of 95° C. for 15 s and 60° C. for 1 min. We employed absolute quantification method and the expression value of each gene was normalized using Actin 13 expression levels. For detailed analysis of genes related to cAmp/Ca²⁺ signaling pathway, RT² Profiler™ PCR array PAMM-066 from SuperArray was used according to manufacturer's protocol. Briefly, total RNA was extracted from P13 wild-type and Satb1 knockout cortexes, RT² First Strand Kit (SuperArray) was used for cDNA synthesis and SuperArray RT² qPCR Master Mix was used to prepare qRT-PCR reactions. Obtained data was analyzed by using software provided by SuperArray. Combination of three different housekeeping genes (Gusb, Hprt, Hsp90ab) was used to normalize the data.

Chromatin immunoprecipitation assay (ChIP) for in vivo DNA binding. Urea-ChIP experiments were performed by following the previously described procedure (Horike 2005, Kohwi-Shigematsu 1998). Briefly, we crosslinked chromatin in one-day-old wild-type brain cells, prepared by a 70 μm strainer (BD Biosciences), by incubating cells in Dulbecco's modified Eagle medium containing 1% formaldehyde for 10 min at 37° C. followed by 2 h at 4° C. We purified crosslinked chromatin from free proteins and RNA by urea gradient centrifugation. We digested the purified chromatin with Sau3AI and precleared it by incubating the chromatin fragments with protein A-Sepharose beads (Sigma) and with preimmune serum. We incubated the precleared chromatin with either preimmune serum or antibody to Satb1 for 4 h at 4° C. and then with protein Sepharose-A beads overnight at 4° C. and finally washed the chromatin fragments on beads four times with 1% NP-40 in phosphate-buffered saline and two times with washing buffer (10 mM Tris-HCl (pH 8.0), 250 mM LiCl, 0.5% NP-40, 0.5% sodium deoxycholate and 1 mM EDTA). We then digested the samples with 250 μg ml⁻¹ proteinase K, treated them at 65° C. for 6 h to reverse crosslinking and subjected them to phenol-chloroform extraction before ethanol precipitation with glycogen. To determine whether any specific DNA sequences were bound to Satb1 in vivo we carried out PCR amplification using these two sets of DNA sequences (from DNA precipitated with antibody to Satb1 or with preimmune serum) as templates. As positive internal control, we used primers (5′-CGTTGGTACTTGTAGGAGCTGAAGC-3′ (SEQ ID NO: 15) and 5′-GGCAGAGATTTGGTGAATGTAAGC-3′ (SEQ ID NO: 16)) to amplify genomic region which was previously identified as Satb1 in vivo binding site by combining urea-ChIP and cloning (T. Kohwi-Shigematsu unpublished data, Kohwi-Shigematsu 1998). For putative Satb1 target genes, we designed the primer sequences mainly focused on the promoter regions of each gene covering 20 kb upstream area and first intron. All primer sequences are available upon request. We resolved PCR products by electrophoresis on 2% agarose gel.

Organotypic slice cultures. Brains from P7 SATB1 knockouts and wild-type littermates were removed quickly, placed into ice-cold dissection media (1× Hanks Balanced salt solution, 10 mM Tris HCl (pH 7.2) in DMEM), cortexes were dissected and cut into small pieces with tissue chopper (McIllwain) and spread onto culture plate inserts (0.4 μm, Millipore) submerged into pre-warmed culture media (25% Horse serum, 0.25× Hanks balanced salt solution, 25 mM Hepes in DMEM). Cortex cultures were maintained for 10 days at 37° C. and 5% CO₂ followed by treatment with 25 μM forskolin to induce elevated levels of cAMP. Treatment with 0.1% DMSO was used as a control (0 h). Treated cultures were collected and total RNA was extracted and followed by cDNA synthesis as described above. qRT-PCR system RT² Profiler™ PCR array PAMM-066 from SuperArray was used to determine the expression levels of 84 cAMP or Ca2+ regulated genes at different time points in wild type and knockout cultures. Obtained data was analyzed by using software provided by SuperArray. Combination of three different housekeeping genes (Gusb, Hprt, Hsp90ab) was used to obtain normalized expression values for each gene. The normalized expression values for each gene at different time points post induction in each sample were divided by normalized expression values at 0 h in that sample and fold induction values were obtained.

### Immediate Early Genes are SATB1 Direct Target Genes

Our Affymetrix expression data revealed an immediate early gene Egr2 to be significantly downregulated in the SATB1-null cortex. We used quantitative real-time PCR (qRT-PCR) to confirm Egr2 expression and also tested expression in wild-type and SATb1-null samples of three additional immediate early genes, Egr1, Fos and Arc, that had been omitted from the list of dysregulated genes due to their p values slightly higher than 0.5. For qRT-PCR, the relative mRNA expression level for each gene was determined using RNA from the cortex isolated from at least 4 different SATB1-null mice and 4 wild-type littermates and the expression level of each gene in SATB1-null sample was normalized to that of wild-type in the same litter. All four immediate early genes were found significantly downregulated in SATB1-null brain by qRT-PCR, indicating this class of genes are preferentially affected by SATB1 ablation. Similarly, we examined 7 genes, including those known to play important roles in neuron or at least expressed in the brain, out of 109 genes significantly dysregulated (>1.4 fold and p<0.5 by Affymetrix). Among which Somatostatiin (Sst), Synaptoporin (Synpr), GABA-A receptor, subunit alpha2(Gabra2), and Retinol binding protein 4(Rbp4) expression were down regulated in the SATB1-null cortex, while PDZ and LIM domain 3(Plim3) and Neuronal pentraxin 2(Nptx2) were upregulated (FIG. 6C). Gapdh and Bdnf were used as negative control genes based on Affymetrix data. Taken together, qRT-PCR confirmed the Affymetrix results and verified that a variety of genes required for brain function are dysregulated in SATB1-null cortex and most importantly, all immediate early genes tested are downregulated in the absence of SATB1.

### Immediate Early Gene Loci are Bound by SATB Family Proteins

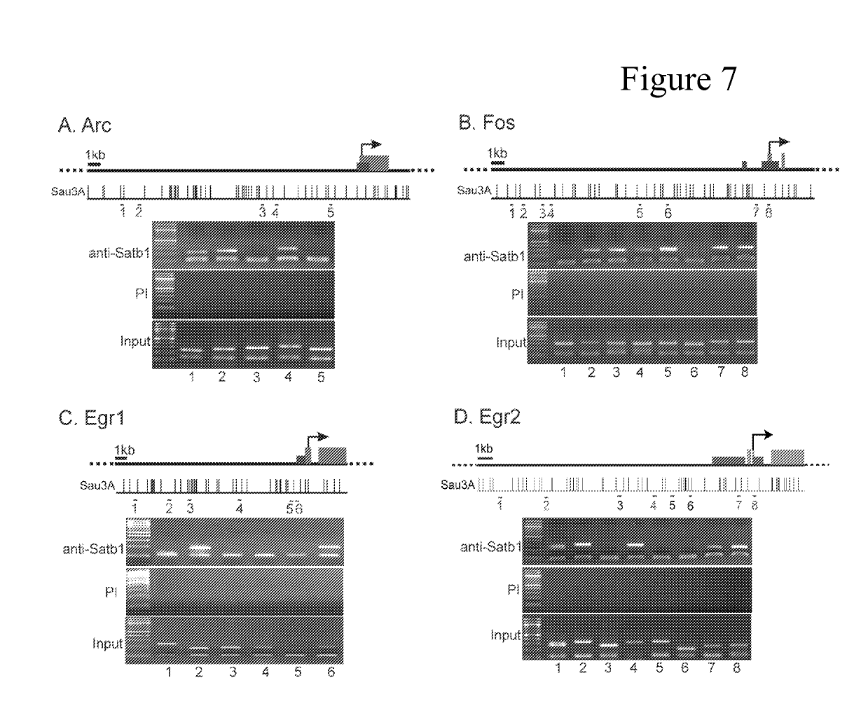

Because immediate early genes are known to play roles in the cellular processes such as learning and memory, we wished to determine whether immediate early genes are direct target genes of SATB family proteins. To test this, we determined their in vivo binding status of the proteins in 1 day old whole brain using the urea-chromatin immunoprecipitation (Urea-ChIP) method and used our rabbit polyclonal antibody against SATB1 (which also detects SATB2) for ChIP. We designed primers within 20-kb upstream sequences of Egr1, Egr2, Arc and Fos gene loci at sequences that exhibit ATC sequence characteristics as potential SATB1-binding sites as well as multiple other sites as controls. Primers were designed similarly for Actin-β and Gapdh loci whose expression in the cortex was similar between SATB1-null and wild-type mice. These primers were used to amplify genomic DNA specifically bound to SATB proteins. We also used primers from different genomic locus which is known to be bound to SATB1 as a positive internal control to normalize sample loading. The experiments revealed that SATB family proteins directly bind all four immediate early gene loci, indicating the direct role of SATB family proteins in immediate early gene expression regulation (FIG. 7). Surprisingly, we detected weak SATB-binding activity within the Actin-β loci. This might reflect this gene may be poised for SATB1 regulation in response to signaling (see below). Under the conditions used, the SATB1 binding was undetectable in the Gapdh loci.

### Example 3

### cAMP-Responsive Genes are Dysregulated in SATB1-Null Cortex

The expression of immediate early genes is regulated by secondary messenger such as cAMP and Ca²⁺. To study the effect of SATB1 on activity-dependent response of the brain, we set out to study cAMP/Ca²⁺ pathway related gene expression in depth. We used the RT² Profiler™ PCR Array, a commercially available qRT-PCR array, that contained primer sets of 84 different genes known to be regulated by cAMP or Ca²⁺ and multiple control genes for normalization. We first compared mRNA expression in 13 day old SATB1-null cortex with the wild-type cortex and found 10 genes either significantly up or down regulated (Table 1). This data indicates that in addition to immediate early genes SATB1 also regulates many other cAMP-responsive genes.

### Example 4

### SATB1 Modulates Activity-Dependent Gene Expression in Mouse Cortex

Upon stimulation in neurons, second messenger pathways are activated that lead to an enhancement in transcription factor activity at gene promoters. A large number of neurotransmitters, hormones and other signaling molecules use cAMP as an intracellular second messenger. In order to examine SATB1's effect on activity dependent gene alteration, we employed forskolin (FK) stimulation on cortical brain culture which increases adenylate cyclase activity in the cell and results in elevates levels of cAMP, thus affecting genes regulated through the cAMP-responsible element (CRE).

We prepared organotypic cortical cultures from 7-days old SATB1-null and wild type littermate pups and added forskolin (25 μM) to the medium 10 days later to induce elevated levels of cAMP. We used the same qRT-PCR system as above (Table I) to determine the expression levels of these genes at early (1 h and 2 h) and later (4 h) time points after stimulation. Each expression level was normalized with average of three house keeping genes ( ). To assess the fold difference in expression upon forskolin stimulation, the ratio of expression level of each gene at a given time point relative to that before activation was plotted for SATB1-null and wild-type brain culture samples. Data shown in FIG. 7 represents the summary of at least 3 different wild type and 3 knockout cultures. Out of 84 genes tested, we found a total of 16 genes to have significantly altered induction pattern in response to forskolin stimulation in SATB1-null cultures compared to wild type cultures. The affected genes include of transcription related genes (4 out of 13), neuropeptides/neurotransmitters (6 out of 21), Ca²⁺-responsive element containing genes (3 out of 10), immune regulation (1 out of 4), cell cycle (1) and SRE-containing gene (1). Although no Ca²⁺ responsible element-containing genes were found altered in the cortex of SATB1-null mouse brain (Table 1), this class of genes were newly identified to possess SATB1-dependent activity upon forskolin stimulation.

There are differences in the patterns of induction between SATB1-null and wild-type cortical cells in culture. For instance, for transcription factor genes, Egr2 and Fosb, exhibit much enhanced induction in expression at the early time point (1 hr) in SATB1-null brain cells compared to wild-type brain cells (FIG. 8A, top). Early responsive genes, Egr1 and Fos, showed virtually identical pattern for gene inducibility upon forskolin stimulation (FIG. 8A, middle), even though we observed that these two gene expression was repressed in vivo without stimulation (Table 1). Other transcription factor genes, Atf3 and Srf, are induced at much higher levels in SATB1-null brain cells at a later time point (4 hr) compared to wild-type brain cells (FIG. 8A, bottom).

Similarly to transcription factor genes, Ca-responsible genes (Calb1, Calr, Calb2), Neurotransmitter genes (Adrb1, Tacr1, Sst, S100g, Sstr2, Penk1), immune-related (Il6), cell cycle (Pmaip1), SRE-containing gene (Thbs1) showed consistently higher levels of inducibility by forskolin stimulation mostly at later time points. These data strongly suggest that in mouse brain, SATB1 is normally regulating gene expression of cAMP/Ca2+ responsible genes to avoid their over-induction of activity-dependent expression.

### Example 5

### Generation of SATB1 Conditional Knockout Mice

We generated straight conditional KO mice SATB1^{Flox/Flox} mice because these mice allow one to generate any mice in which SATB1 is deleted from any other tissue by just breeding with transgenic mice harboring cell-type specific gene promoter linked Cre recombinase gene. Breeding with Synapsin-Cre mice allowed the deletion of SATB1 from Synapsin expressing cells, such as neurons. A final mouse line, SATB1^{Flox/Flox}; Syn-Cre mice, was also generated using the following steps and constructs as shown in Figure X.

A targeting construct (b) shown in FIG. 9A panel was constructed and mice harboring a conditional allele (b) were generated. Such heterozygous mice containing a single conditional allele were bred to produce mice homozygous for conditional allele (designated as SATB1^{Flox/Flox}). As shown in FIG. 9B panel, SATB1^{Flox/Flox} mice were bred with mice containing Synapsin promoter driven Cre recombinase (Syn-Cre) as a transgene under SATB1 wild-type background (SATB1^{+/+}; Syn-Cre/+). Mice having genotype SATB1^{Flox/+}; Syn-Cre/+ will be selected to be bred with SATB1^{Flox/Flox} mice to produce mice (SATB1^{Flox/Flox}; Syn-Cre/+) in which SATB1 was deleted from Synapsin gene expression neurons a shown in c) under panel A.

### Example 6

### SATB1 Knockout Mice have Altered Aggression, Inhibition and Anxiety Levels

For normal human social behavior, it is important to read environmental and social clues correctly, respond to them correctly and learn from your experiences. Dysfunction in regulation of any of these processes may result in psychological disorders. A variety of genes, including immediate early genes, neurotransmitters and their receptors, work together to fine tune these processes.

As shown in the previous Examples, SATB1 plays a key role in normal regulation of gene expression post-natally. Thus, abnormal SATB1 expression levels could play a role in abnormal behavior and psychological disorders.

Elevated Plus Maze. The elevated plus maze (EPM) was used to examine fear and anxiety in SATB1 conditional knockout mice. The maze was comprised of two open (30×5×0.25 cm) and two closed (30×5×6 cm) arms, which extended from a common central platform (5×5 cm). The apparatus was constructed from black Plexiglas and was elevated to a height of 60 cm above the floor level. All testing was conducted under dim red light. Mice were individually placed on the central platform of the maze facing a random corner between an open and a closed arm and allowed to explore the maze for 5 min. The EPM was attached to a camera and distance traveled, number of entries into each arm and into the central platform, time in open vs. closed arms and latency to first arm entry were digitally recorded. Fearful anxious animals spent more time in the closed arms of the maze

The behavior of control and SATB1-CKO mice was examined in elevated plus maze test (EPM). This test relies on the natural conflict between tendency of mice to explore a novel environment and the tendency to avoid a brightly lit, elevated, open area (Montgomery 1958). Testing in the EPM allows assessment of anxiety (number of entries and time spent in open arms), and activity (entries and distance traveled in closed arms) during the same trial (File 2001). Referring now to FIG. 10, SATB1-CKO mice showed higher numbers of open arm entries than control mice (11 vs. 7 entries, respectively). SATB1-CKO mice made the same number of entries into open and closed arms indicating no preference for closed arms. Control mice showed clear preference for closed arms, making about two times less of entries into open arms than into closed arms (7 vs. 14 entries, respectively). SATB1-CKO mice spent about the same amount of time in both open (41% out of total time) and closed arms (45%) and stayed less time in center area (14%). Control mice clearly preferred spending their time in closed arms (80% out of total) while the permanence time in open arms and center area was much less (11% and 9%, respectively). Both control mice and CKO mice exhibited similar overall mobility during this test. Therefore, the test result is not due to hyperactivity of CKO mice. These results indicate that SATB1-CKO mice have significantly reduced anxiety levels when compared to controls. We did not see any significant difference in the average velocity or total distance traveled between SATB1-CKO and control mice indicating similar activity levels in this test.

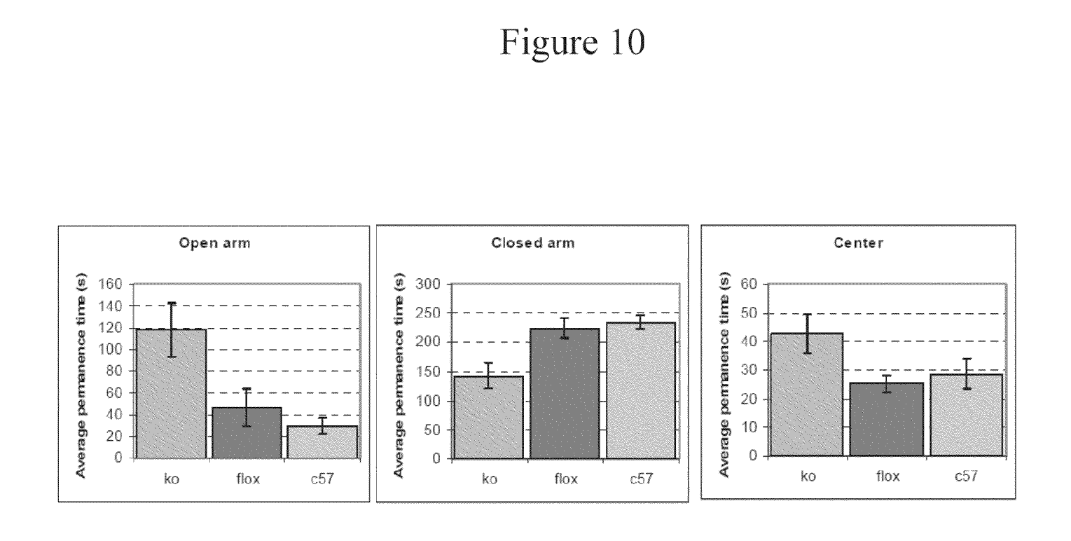

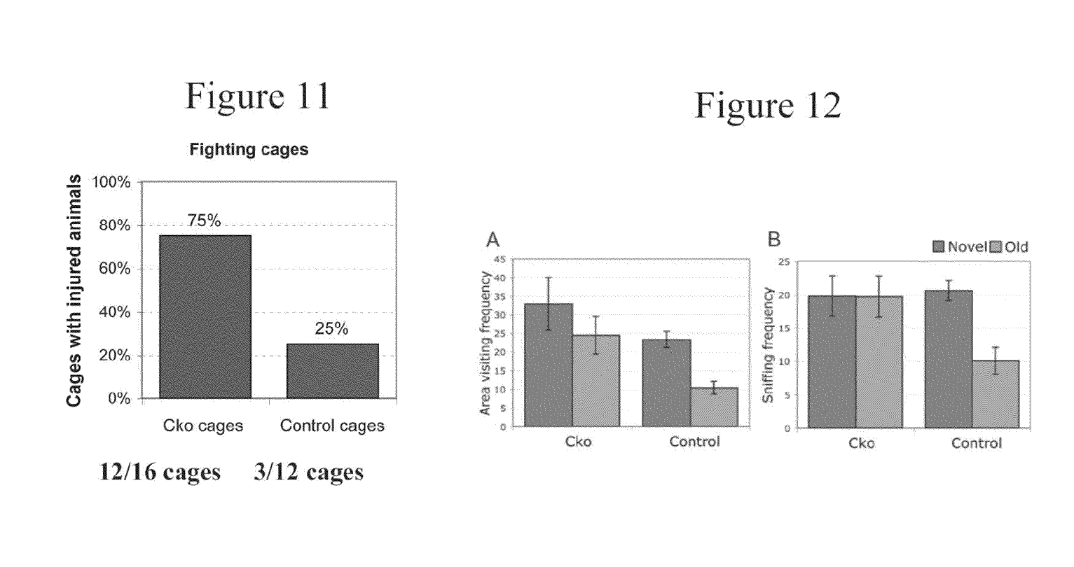

Aggression observation. We noticed that in our transgenic mouse colony, there is a high incidence of injuries from fighting. Usually, all mice besides one aggressive one were injured. All incidences of injuries were recorded and total of 16 cages containing conditional SATB1 knockout and 13 cages containing only control mice were analyzed. We concluded that there is much higher injury rate in cages containing among other mice also SATB1 knockout (75%) than in cages containing only control mice (25%) (FIG. 11). In 7 out of 11 cages (64%), the uninjured mouse, which was probably also the aggressive one, was conditional SATB1 knockout.

Object novelty. The object novelty test consists of two parts. One the first day of testing mouse is placed for 5 min into empty testing apparatus for habituation trial. On the next day animal is placed in the apparatus with the two objects. The animal is given 5 min to investigate the objects. The animal is removed from the apparatus and placed into holding cage. One of the objects is replaced with a novel object in the testing apparatus. The animal is returned into testing apparatus and has 5 min to investigate the objects. Mouse behavior near novel object and near familiar object is recorded. Time spent in the area, times visited and sniffing frequency are recorded. We observed that conditional SATB1 knockout mice visited areas with novel or old object with the same frequency while control mice clearly preferred the area with novel object (FIG. 12). Also, conditional SATB1 knockout mice sniffed both novel and old object the same amount of time while control mice were clearly more interested in novel object by sniffing it about twice as much as familiar object.

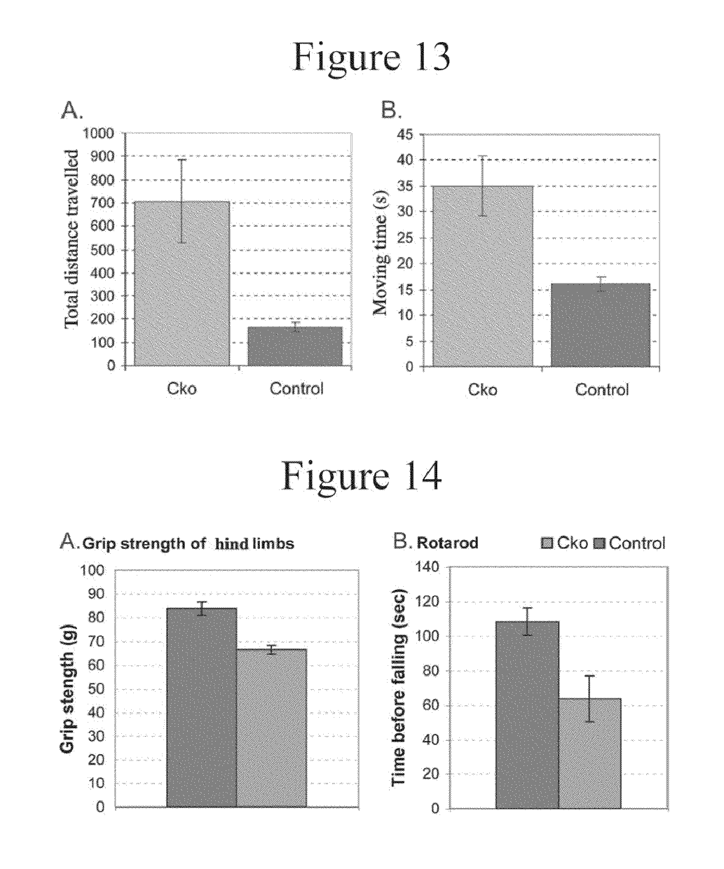

Integra. The integra is used to observe general mouse behavior. Mice are individually placed into testing chamber and the behavior is videotaped for 26 hours. The general activity (distance traveled, moving time, number of moving episodes etc.) and also any abnormal behavior (stereotypic behavior) are recorded. We observed that the general activity is much higher in conditional SATB1 knockout mice compared to control mice. The knockout mice traveled more than 4 times longer distance than control mice (FIG. 13). Although the number of moving episodes was similar in mice from both genotypes, the conditional SATB1 knockouts moved about 2 times longer in each episode.

Grip strength. The grip strength meter allows the study of neuromuscular functions in mice by determining the maximum force displayed by an animal. The grip strength of mouse front limbs and hind limbs was recorded. We observed that the grip strength of hind limbs of conditional SATB1 knockout mice is slightly weaker than in control animals (FIG. 14A).

Rotarod. The rotarod is used to observe motor coordination and balance in mouse. The mouse was placed on a rotating cylinder and the number of falls and flips were recorded in two 2 min trials If the mouse fell it was placed back onto the cylinder for the remaining 2 min trial. We observed that the average time for mice being able to stay on rotating cylinder was almost two times shorter for conditional SATB1 knockout mice than for control mice (FIG. 14B). Also, about 67% of conditional SATB1 knockout mice were falling off from the rotarod at some point during testing while only 20% of control mice ever fell. These results indicate that conditional SATB1 knockout mice have a problem with motor coordination and/or balance.

### Example 7

### SNP Genotyping for SATB1

SNP genotyping for SATB1 can be carried out by steps 1) isolating the DNA samples from the organisms/cells of different individuals and 2) amplifying the gene of interest from each sample using gene specific primers.

Software such as Primer Plex (PREMIER BioSoft International, Palo Alto, Calif.) is used to design oligonucleotides for primer extension assays on a LUMINEX platform (Luminex Corp., Austin, Tex.) Using oligonucleotides for multiplex analysis on suspension array systems such as LUMINEX100 offers a versatile platform for multiplex nucleic acid detection in the 96-well format. Primer extension is a two step process that first involves the hybridization of primers used for primer extension method that have 3′ bases which are complementary to each of the SNP alleles being interrogated, followed by a ‘mini-sequencing’ reaction, in which DNA polymerase extends the hybridized primer by adding a base that is complementary to the SNP nucleotide. If the target DNA contains SNPs complementary to the primer's 3′ base, the primer will completely hybridize to the target DNA. This is detected by TAG (Luminex Platform) specific to the mutation. If the target DNA does not contain SNP complementary to the primer's 3′ base, there will be a mismatch at the 3′ end of the primer and DNA polymerase will not be able to extend the 3′ end of the primer (Goelet, p et al., Mol Cell Probes. 1999 April; 13(2):81-7; Syvanen 2001). Details on PrimerPlex is described in <http://www.premierbiosoft.com/primerplex/index.html>.

All SNPs that are reported in NCBI dbSNP <http://www.ncbi.nlm.nih.gov/>, USCS genome bioinformatics <http://genome.ucsc.edu/> and any novel SNPs found will be made into the Luminex 100 array platform to provide an array for detecting SATB1 and SATB2 SNPs in an individual.

### Example 8

### Delivery of SATB1 and/or SATB1 Probes for Imaging and Therapeutic Purposes

Sample surgery procedures for a subject (e.g., a canine or human). One skilled in the art should know how to adjust the dosages and procedures. Prior to induction of anesthesia, subjects are pre-medicated with oxymorphone (0.04-0.06 mg/kg, SC), diazepam (0.03-1.0 mg/kg IM), and atropine (0.02-0.04 mg/kg SC), induced with thiopental (10.0 mg/kg IV), and intubated. Anesthesia is maintained with isoflurane (1.5% in oxygen) and a PaCO.sub.2 is maintained between 30-35 mmHg using positive pressure ventilation. Body temperature is maintained between 37.5-39.0° C. with the aid of circulating air/water blankets. An intravenous cephalic catheter, an indirect pressure cuff, and EKG leads are placed for monitoring of mean arterial blood pressure and EKG while under anesthesia. Blood gasses, blood glucose, and electrolytes are monitored every 30 to 60 minutes during anesthesia. Intravenous fluid administration (Lactated Ringer's solution, 10-12 ml/kg/hr) is continuous throughout the anesthetic period. Temperature, respiratory rate, heart rate, mucous membrane color, and mentation are monitored every 10 minutes during anesthetic recovery. When subjects are fully recovered, a neurologist assesses neurological signs during post-recovery phase.

The subject's head is first placed in a MRI compatible stereotactic frame prior to obtaining an initial baseline MRI that determines the location of the guide cannula assembly. Surgical exposure for placement of cannulae involved a midline skin incision and retraction of the temporalis muscle to expose the cranium over the cannula entry site. Using a Hall air drill, a small burr hole is made in the skull to expose the dura over the infusion site. A 21-gauge needle is used to penetrate the dura to expose the cortex above each infusion site and additional burr holes are created adjacent to each infusion site to position brass set screws. Using a stereotactic tower, each guide cannula assembly is stereotactically lowered into the burr hole, the hole filled with acrylic, and the cannula assembly secured using dental acrylic. Once the guide cannula is secured, additional acrylic can be applied to bond the guide to several screws positioned on the skull. The wound site is closed in anatomical layers over the guide cannula. Each subject is monitored for full recovery from anesthesia, placed on antibiotics and observed twice daily for 5 days following surgery.

Liposome preparation: For example, see Noble et al., Cancer Res. Mar. 1, 2006; 66(5):2801-6. 1,1′-dioctadecyl-3,3,3,3′-tetramethylindocarbocyanine-5,5′-disulfonic acid (DiIC.sub.18(3)-DS) is obtained from Molecular Probes (Eugene, Oreg.), 1-2-dioleoyl-3-sn-glycerophospho-choline (DOPC) and poly(ethylene glycol)-1,2-distearoyl-3-sn-phosphoethanolamine (PEG-DSPE) from Avanti Polar Lipids (Alabaster, Ala.), and cholesterol (Chol) from Calbiochem (San Diego, Calif.). DOPC and Chol (molar ratio 3:2), PEG-DSPE (5 mol %) and optional DiIC.sub.18(3)-DS (0.2 mol %) are mixed in chloroform and dried by rotary evaporation. For MRI studies, liposomes are passively loaded with Gd (Omniscan™) (GD-liposomes). The lipid film is rehydrated in Gd solution (250 mM), followed by 6 successive cycles of rapid freezing-thawing, and subsequently extruded through polycarbonate filters with defined pore sizes (5.times.0.2.mu.m, 5.times.0.05.mu.m), yielding liposomes of about 80 nm diameter as determined by dynamic light scattering. Unencapsulated Gd is removed using a Sephadex G-75 size exclusion column (Pharmacia, Piscataway, N.J.), followed by extensive dialysis against HEPES buffered saline (HBS) (pH 6.5). Liposome concentration is measured by standard phosphate analysis and adjusted to a normalized level of phospholipid for all experiments.

To image and deliver normal SATB1 to a subject intrastriatally, liposomes are then loaded with normal SATB1 protein or a viral vector containing the SATB1 cDNA. Liposomes (20 mM phospholipids) are optionally labeled or loaded with fluorescent dyes (either rhodamine or Dil-DS) or other imaging label.

A combination CED of liposomal SATB1 and labeled liposomes are administered and infused by CED at a volume of either 33 μl or 99 μl into the cerebral cortex, hippocampus or amygdala in a subject. Real-time monitoring of liposome distribution is obtained in the cerebral cortex, hippocampus or amygdala. MR images are obtained at approximately 10-minute intervals during the infusions. These real-time images detect liposomal distribution from approximately 10 to 20 minutes after initiation of infusion and should clearly detect the enlargement of distribution area during infusion.

Alternatively, lentiviral-based vectors such as HIV and FIV are constructed with normal SATB1 or SATB1 cDNA and the vector is delivered intrastriatally by injection to the subject.

The present examples, methods, procedures, specific compounds and molecules are meant to exemplify and illustrate the invention and should in no way be seen as limiting the scope of the invention. Any patents, publications, publicly available sequences mentioned in this specification are indicative of levels of those skilled in the art to which the invention pertains and are hereby incorporated by reference to the same extent as if each was specifically and individually incorporated by reference.

TABLE I
SATB1-dependent altered genes within 81 cAMP or
Ca2+ responsive genes in 13 days cerebral cortex

| | | Fold | Function | |
|---|---|---|---|---|
| Gene | Gene name | Difference | (Location) | Enhancer |
| Fosb | FBJ osteosarcoma | −2.00 | T (n) | SRE |
| | oncogene B | | | |
| Egr2 | Early growth response 2 | −1.64* | T (n) | CRE, SRE |
| Fos | FBJ osteosarcoma | −1.61* | T (n) | CRE, SRE |
| | oncogene | | | |
| Cyr61 | Cysteine-rich protein 61 | −1.56 | G (s) | SRE |
| Th | Tyrosine hydroxylase | −1.54 | N (c, n) | CRE |
| Atf3 | Activating transcription | −1.43 | T (n) | CRE |
| | factor 3 | | | |
| Areg | Amphiregulin | −1.41 | G (s) | CRE |
| Prl | Prolactin | −1.37 | N (s) | CRE |
| Egr1 | Early growth response 1 | −1.34* | T (n) | CRE, SRE |
| Sst | Somatostatin | −1.33 | N (s) | CRE |
| Gcg | Glucagon | 1.42 | N (s) | CRE |
| Plf | Proliferin | 1.43 | N (s) | CRE |
| S100a8 | S100 Ca²⁺-binding | 1.46 | N (c, n) | CRE |
| | protein A8 | | | |
| S100a9 | S100 Ca²⁺-binding | 1.69 | N (c, n) | CRE |
| | protein A9 | | | |
| Tnf | Tumor necrosis factor | 2.07* | G (m, s) | CRE |
| I16 | Interleukin 6 | 2.19* | I (s) | CRE |
| I12 | Interleukin 2 | 2.20 | I (s) | CRE |

N: Neuropeptide/neurotransmitters; T: transcription regulation; G: growth factors; I: immune regulation; (n): nucleus; (s): secreted; (m): membrane; (c): cytoplasm. CRE: cyclic AMP response element containg gene; SRE: serum response element.or SRE-like element containg gene;
*P < 0.05 (Student's t-test with 5 independnet experiements).

What is claimed is:

1. A conditional homozygous Satb1-null non-human animal comprising a genome having a functionally disrupted Satb1 gene, wherein the Satb1 gene is disrupted only in neurons in a developmental stage specific manner or brain subregion-specific manner.

2. The animal of claim 1 wherein the animal is a mouse.

3. The animal of claim 2 wherein the animal is a Satb1^{flox/flox}: Synapsin-promoter driven-Cre recombinase and/or a Satb1^{flox/flox}: CamKII-promoter driven-Cre recombinase mouse.

4. The animal model in claim 2 which find use in studies to confirm association and causative effect of Satb1 depletion on neurological dysfunction or disease.

5. A synthetic polynucleotide having a sequence encompassing a single nucleotide polymorsphism (SNP) or variant sequence in SEQ ID NO: 3 and/or SEQ ID NO:7 for use in detecting the presence of a wild-type or a rare allele of said SNPs, wherein the presence of said SNP is associated with a neurological disorder or disease.

6. An array of oligonucleotide probes to detect a single nucleotide polymorphism in variant SATB1 and/or SATB2 in patient sample, comprising at least one oligonucleotide representing wild type SATB1 and/or SATB2 and one oligonucleotide representing variant SATB1 and/or SATB2, wherein the detection of a variant SATB1 and/or SATB2 indicates the patient may have an associated neurological disfunction or disease.

7. The array of claim 6, wherein the array comprising oligonucleotide probes representing all publicly available SATB1 and SATB2 SNPs and novel SATB1 and SATB2 SNPs found, thereby providing an array platform for diagnostic detection.

8. A method for determining the genetic status of an individual, comprising:
detecting the presence of one of more single nucleotide polymorphisms (SNPs) in SATB1 or SATB2 in the DNA or RNA of the individual; and
whereby
the presence of one or more SNPs indicates neurological dysfunction in the individual.

9. The method of claim 9, wherein the SNP in SATB1 or SATB2 is detected by an antibody or by an array of probes.

10. A therapeutic composition for restoring impaired SATB1 or SATB2 protein and function in the brain.

11. The composition of claim 10, wherein the composition is an isolated polynucleotide having at least 70% homology to the cDNA SATB1 or SATB2 sequence selected from the group consisting of SEQ ID NO: 3 and SEQ ID NO: 7.

12. The composition of claim 11, wherein the composition further comprising a vector for gene therapy for delivery to the brain of a subject.

13. A method of using SATB1 or SATB2 to identify crucial genes which, when mutated, lead to neurological disorders, wherein said identification of genes is based on (a) chromatin immunoprecipitation of genes where SATB1 or SATB2 binds in vivo, and (b) expression profiles that show dependency on SATB1 or SATB2.

14. The method of claim 13, wherein the genes which are directly regulated by SATB1 or SATB2 comprising the genes found in Table 1.
